(19) United States
(12) Patent Application Publication
(10) Pub. No.: US 2024/0229128 A1
(43) Pub. Date: Jul. 11, 2024

(54) METHODS FOR IMPROVED T CELL RECEPTOR SEQUENCING

(71) Applicant: ADOC SSF, LLC, South San Francisco, CA (US)

(72) Inventors: Benjamin T.K. YUEN, South San Francisco, CA (US); Saparya NAYAK, South San Francisco, CA (US); Songming PENG, South San Francisco, CA (US); Stefanie MANDL-CASHMAN, South San Francisco, CA (US)

(73) Assignee: ADOC SSF, LLC, South San Francisco, CA (US)

(21) Appl. No.: 18/536,752

(22) Filed: Dec. 12, 2023

Related U.S. Application Data
(63) Continuation of application No. PCT/US22/34005, filed on Jun. 17, 2022.
(60) Provisional application No. 63/212,286, filed on Jun. 18, 2021.

(51) Int. Cl. C12Q 1/6869; C12N 15/10; C12Q 1/6806

(52) U.S. Cl. CPC C12Q 1/6869; C12N 15/1096; C12Q 1/6806

(57) ABSTRACT

The present disclosure relates to methods for sequencing and identifying T cell receptors (TCR). The methods include the use of template-switching oligonucleotides (TSOs) and as well as result in improving yield and reducing complexity compared to standard methods.

### CROSS-REFERENCE TO RELATED APPLICATIONS

This application is a Continuation Application of International Patent Application No. PCT/US22/34005, filed Jun. 17, 2022, which claims priority to U.S. Provisional Application No. 63/212,286, filed Jun. 18, 2021, the content of which is incorporated by reference in its entirety.

### SEQUENCE LISTINGS

The instant application contains a Sequence Listing which has been submitted electronically and is hereby incorporated by reference in its entirety. Said Sequence Listing, created Dec. 12, 2023, is named 087520.0337.xml and is 74,376 bytes in size.

### FIELD OF INVENTION

The present disclosure provides methods for preparing complementary deoxyribonucleic acid (cDNA) molecules comprising full-length T cell receptor (TCR) sequences and determining the nucleic acid sequence of those TCRs.

### BACKGROUND

Accurately identifying full-length TCR sequences is an important step for a variety of applications, including, but not limited to, the development of personalized medicine and adoptive cell therapies, where the loss of TCR diversity can result in the failure to identify therapeutically effective molecules. Conventional methods for cloning TCRβ are based on a combination of reverse transcription-polymerase chain reaction (PCR) followed by a first and second round of nested PCR reactions which amplify the VJ segment of the TCRα chain and the VDJ segment of the TCRβ chain. These segments can then be analyzed in silico to allow for the reconstruction of the full-length TCR sequence. A substantial proportion of the TCR sequences reconstructed in this fashion, however, exhibit ambiguous results. This ambiguity can be related, for example, to the use of nested primers that mask TCR sequence variations during the amplification steps. Thus, there remains a need for the development of relatively low-cost, high throughput sequencing technologies capable of providing accurate full-length TCR sequences.

### SUMMARY

The present disclosure provides methods for preparing cDNAs comprising full-length TCR sequences and determining the nucleic acid sequence of those TCRs. In certain non-limiting embodiments, the present disclosure provides a method of preparing a deoxyribonucleic acid (DNA) comprising a full-length T cell receptor (TCR) sequence. In certain embodiments, the method comprises: obtaining a TCR complementary DNA (cDNA) sequence by combining a ribonucleic acid (RNA) molecule with a cDNA synthesis primer complementary to a region of the RNA molecule 3′ to the TCR coding sequence, a template-switching oligonucleotide (TSO), and a reverse transcriptase under conditions sufficient for the reverse transcriptase to produce the cDNA sequence; and preparing a DNA comprising a full-length TCR sequence by combining the cDNA with a first set of amplification primers comprising an outer primer annealing to the TSO sequence and a primer annealing to a TCR constant sequence and a polymerase under conditions sufficient for the polymerase to produce a DNA comprising a full-length TCR sequence. In certain embodiments, the method further comprises sequencing the DNA comprising a full-length TCR sequence.

In certain embodiments, the RNA molecule is extracted from a sample comprising T cells. In certain embodiments, the sample is collected from a subject. In certain embodiments, the cDNA synthesis primer is an oligo-dT primer. In certain embodiments, the oligo-dT primer comprises a polynucleotide having a sequence set forth in SEQ ID NOs: 1 or 2. In certain embodiments, the TSO comprises an amplification primer site, a barcode, and a unique molecular identifier (UMI). In certain embodiments, the TSO comprises a polynucleotide having a sequence set forth in any one of SEQ ID NOs: 3-5. In certain embodiments, the reverse transcriptase is a template-switching reverse transcriptase.

In certain embodiments, the outer primer comprises a polynucleotide having a sequence set forth in SEQ ID NOS: 6. In certain embodiments, the inner primer comprises a polynucleotide having the sequence set forth in SEQ ID NO: 11. In certain embodiments, the primer annealing to a TCR constant sequence comprises a polynucleotide having the sequence set forth in SEQ ID NOS: 7, 8, 12, and 13. In certain embodiments, the DNA comprising a full-length TCR sequence is further amplified using a second set of amplification primers, wherein one or both of the primers comprise a barcode sequence. In certain embodiments, the full-length TCR receptor comprises a TCRα chain and/or a TCRβ chain. In certain embodiments, the full-length TCR receptor comprises a TCRγ chain and/or a TCRδ chain.

In certain embodiments, the method comprises an extension phase from about 20 seconds to about 90 seconds. In certain embodiments, the amplification primers comprise a forward primer at a concentration from about 0.1 μM to about 0.6 μM.

In certain non-limiting embodiments, the present disclosure also provides a cDNA library produced by the methods disclosed herein.

In certain non-limiting embodiments, the present disclosure further provides a method of analyzing single T cells to determine the nucleic acid sequence of a full-length TCR sequence. In certain embodiments, the method comprises sorting single T cells from a sample comprising a plurality of T cells; preparing a DNA comprising a full-length TCR sequence, comprising: obtaining a TCR complementary DNA (cDNA) sequence by combining a ribonucleic acid (RNA) molecule with a cDNA synthesis primer complementary to a region of the RNA molecule 3′ to the TCR coding sequence, a template-switching oligonucleotide (TSO), and a reverse transcriptase under conditions sufficient for the reverse transcriptase to produce the cDNA sequence; and preparing a DNA comprising a full-length TCR sequence by combining the cDNA with a first set of amplification primers comprising an outer primer annealing to the TSO sequence and a primer annealing to a TCR constant sequence and a polymerase under conditions sufficient for the polymerase to produce a DNA comprising a full-length TCR sequence; and sequencing the DNA comprising a full-length TCR sequence. In certain embodiments, the sample is collected from a subject.

In certain embodiments, the cDNA synthesis primer is an oligo-dT primer. In certain embodiments, the oligo-dT primer comprises a polynucleotide having a sequence set forth in SEQ ID NOs: 1 or 2. In certain embodiments, the TSO comprises an amplification primer site, an identification tag, and a unique molecular identifier (UMI). In certain embodiments, the TSO comprises a polynucleotide having a sequence set forth in any one of SEQ ID NOs: 3-5. In certain embodiments, the reverse transcriptase is a template-switching reverse transcriptase. In certain embodiments, the outer primer comprises a polynucleotide having the sequence set forth in any one of SEQ ID NO: 6. In certain embodiments, the inner primer comprises a polynucleotide having the sequence set forth in SEQ ID NO: 11. In certain embodiments, the primer annealing to a TCR constant sequence comprises a polynucleotide having the sequence set forth in any one of SEQ ID Nos. 7, 8, 12, and 13. In certain embodiments, the amplification primers comprise a barcode sequence.

In certain embodiments, the full-length TCR receptor comprises a TCRα chain and/or a TCRβ chain. In certain embodiments, the full-length TCR receptor comprises a TCRγ chain and/or a TCRδ chain. In certain embodiments, the method comprises an extension phase from about 20 seconds to about 90 seconds. In certain embodiments, the amplification primers comprise a forward primer at a concentration from about 0.1 μM to about 0.6 μM.

In certain embodiments, the method further comprises analyzing the whole transcriptome of the single T cells. In certain embodiments, the method further comprises analyzing somatic mutation or genetic polymorphisms of the single T cells.

In certain embodiments, the sorting comprises contacting the sample with of a plurality of peptide/MHC complexes, wherein each peptide/MHC complex comprises an associated barcode. In certain embodiments, the method further comprises sequencing the barcode associated with each peptide/MHC complex. In certain embodiments, the method further comprises determining a ratio of a first barcode associated with a first peptide/MHC complex and a second barcode associated with a second peptide/MHC complex. In certain embodiments, the method further comprises determining antigen specificity of the T cell based on the ratio of the first barcode and the second barcode.

### BRIEF DESCRIPTION OF THE DRAWINGS

FIGS. 1A and 1B illustrate an exemplary SMART-TCR approach to generating a sequence of a full-length TCR transcript. FIG. 1A shows a general overview of the process of creating a cDNA comprising 5′ and 3′ amplification sequences from a TCR mRNA template. FIG. 1B shows a detailed description of the process for amplification of the full length TCR sequence from the cDNA created in FIG. 1A.

FIG. 2 illustrates the TCR recovery rate, the Signal:Noise ratio, and the average NeoID reads obtained with the SMART-TCR approach and the conventional methods. NeoID: unique oligonucleotide sequence labeling a unique pHLA (peptide-HLA) tetramer. Signal:Noise ratio: estimate of the specificity of a TCR for a pHLA calculated by dividing the reads for a unique pH LA tetramer by the reads identified for a different pHLA tetramer. Clinical imPACT Approach, control set of nested, multiplex primer reaction for the TCR commonly used in the art.

FIG. 3 illustrates the TCR recovery rate, the Signal:Noise ratio, and the average NeoID reads obtained with the SMART-TCR approach in different experimental conditions. Clinical TCR Approach: control set of nested, multiplex primer reaction for the TCR commonly used in the art. SMART-TCR Cleanup RT: bead purification done on the RT product prior to TCR amplification (outlined in FIG. 1B). SMART-TCR Enzyme Spikein: addition of new polymerase enzymes into the RT reaction mixture to promote TCR template formation. SMART-TCR 1.5× NeoID: addition of increased amounts of NeoID primer to increase the Signal:Noise ratio and NeoID Reads. SMART-TCR Low Extension: reduction of the extension time for TCR amplification to promote smaller product formation (such as TCR or NeoID products).

FIGS. 4A-4B illustrate the effects of lowering extension and increasing concentration of primers on TCR recovery rate, Signal:Noise ratio, and average NeoID reads. FIG. 4A shows the TCR recovery rate. FIG. 4B shows Signal:Noise ratio, and average NeoID reads.

FIGS. 5A-5B illustrate the effects of lowering extension and increasing concentration of NeoID primers on TCR recovery rate, Signal:Noise ratio, and average NeoID reads in two distinct samples (LP356 and LP169). FIG. 5A shows TCR recovery rate. FIG. 5B shows Signal:Noise ratio, and average NeoID reads. NeoID: unique oligonucleotide sequence labeling a unique pHLA (peptide-HLA) tetramer. Signal:Noise ratio: estimate of the specificity of a TCR for a pHLA calculated by dividing the reads for a unique pHLA tetramer by the reads identified for a different pHLA tetramer. Clinical imPACT Approach: control set of nested, multiplex primer reaction for the TCR commonly used in the art. SMART-TCR 1.5×NeoID 3 mins Extension, variation in the SMART-TCR process where NeoID primers are increased along with decreasing the extension time of the reaction to 3 minutes. SMART-TCR 2×NeoID 1.5 mins Extension: variation in the SMART-TCR process where NeoID primers are increased along with decreasing the extension time of the reaction to 1.5 minutes. SMART-TCR 2×NeoID 1.5 mins Extension Low 5′primer: variation in the SMART-TCR process where NeoID primers are increased along with decreasing 5′ primers (outlined in FIG. 1B) and the extension time of the reaction to 1.5 minutes.

FIG. 6 illustrates that the SMART-TCR approach yields comparable HLA-matched comPACT distribution to clinical imPACT approach.

### DETAILED DESCRIPTION

The present disclosure provides methods and compositions for preparing complementary deoxyribonucleic acid (cDNA) molecules comprising full-length T cell receptors (TCRs). The present disclosure is based, in part, on the discovery of reagents and methods for sequencing TCR genes and therefore profiling T cells using multiple amplifications and deep sequencing. Finally, the present disclosure also provides methods for producing adoptive cell therapies (e.g., T cell products) using the methods and compositions disclosed herein. Non-limiting embodiments of the present disclosure are described by the present description and examples. For purposes of clarity of disclosure and not by way of limitation, the detailed description is divided into the following subsections:
1. Definitions;
2. Methods of Preparing DNA;
3. Methods of Analyzing T cells;
4. Compositions and Kits;
5. T Cell Products; and
6. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Concise Medical Dictionary, edited by Law and Martin, Oxford University Press, 2020; A Dictionary of Biology, edited by Hine, Oxford University Press, 2019; A Dictionary of Chemistry, edited by Law and Rennie, Oxford University Press, 2020; Oxford Dictionary of Biochemistry and Molecular Biology, edited by Cammack, Atwood, Campbell, Parish, Smith, Vella, and Stirling, Oxford University Press, 2006; Paul, William. 2013. Fundamental Immunology. Philadelphia, PA: Wolters Kluwer Health/Lippincott Williams & Wilkins; and Wong, Lee-Jun C. 2013. Next Generation Sequencing: Translation to Clinical Diagnostics. New York, NY: SpringerLink. As used herein, the following terms have the meanings ascribed to them below, unless specified otherwise.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes”, “including” and the like.

As used herein, the use of the word “a” or “an” when used in conjunction with the term “comprising” in the claims and/or the specification can mean “one,” but it is also consistent with the meaning of “one or more,” “at least one,” and “one or more than one.”

Unless specifically stated or otherwise apparent from context, as used herein the term “about” or “approximately” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Alternatively, the term can mean within an order of magnitude, preferably within 5-fold, and more preferably within 2-fold, of a value.

As used herein, the terms “polynucleotide” and “nucleic acid” are used interchangeably and include any compound and/or substance that comprises a polymer of nucleotides. Each nucleotide is composed of a base, specifically a purine or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Polynucleotide refers to any DNA (including but not limited to cDNA, ssDNA, and dsDNA) and any RNA (including but not limited to ssRNA, dsRNA, and mRNA) and further includes synthetic forms of DNA and RNA and mixed polymers comprising two or more of these molecules. The polynucleotide may be linear or circular. In addition, the term polynucleotide includes both, sense and antisense strands, as well as single-stranded and double-stranded forms. The polynucleotide can contain naturally occurring or non-naturally occurring nucleotides. Examples of non-naturally occurring nucleotides include modified nucleotide bases with derivatized sugars or phosphate backbone linkages or chemically modified residues. Polynucleotides encompass DNA and RNA molecules that are suitable as a vector for direct expression of a polypeptide of the invention in vitro and/or in vivo.

The terms “polypeptide” and “protein” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residues is an artificial chemical mimetic of a corresponding naturally occurring amino acid, as well as to naturally occurring amino acid polymers and non-naturally occurring amino acid polymers.

The term “percent sequence identity.” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent sequence identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “primer.” as used herein, generally refers to an oligonucleotide molecule, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process may be determined by the sequence of the template polynucleotide. Primers are extended by a polymerase. Primers are generally of a length compatible with their use in synthesis of primer extension products and are usually in the range of between about 8 to about 100 nucleotides in length. A “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of nucleic acid synthesis.

The terms “amplifying,” or “amplification,” as used herein, generally refer to the process of synthesizing nucleic acid molecules that are complementary to one or both strands of a template nucleic acid. Amplifying a nucleic acid molecule may include denaturing the template nucleic acid, annealing primers to the template nucleic acid at a temperature that is below the melting temperatures of the primers, and enzymatically elongating from the primers to generate an amplification product. In certain embodiments, the denaturing, annealing and elongating steps are performed multiple times such that the amount of amplification product is increasing. Amplification typically requires the presence of deoxyribonucleoside triphosphates, a polymerase enzyme and an appropriate buffer and/or co-factors for optimal activity of the polymerase enzyme. The term “amplification product” refers to the nucleic acids, which are produced from the amplifying process as defined herein.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single-stranded DNA). Sequencing can be performed by various systems currently available, for example, but without limitation, a sequencing system by Illumina®, Pacific Biosciences (PacBio®), Oxford Nanopore), or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In certain embodiments, such systems provide sequencing reads (also “reads” herein). A read may include a string of nucleic acid bases corresponding to a sequence of a nucleic acid molecule that has been sequenced. The term “sequence read abundance,” as used herein, generally refers to the number of times a particular sequence or nucleotide is observed in a collection of sequence reads. The term “sequencing,” as used herein, generally refers to a method by which the identity of consecutive nucleotides (e.g., the identity of at least 10, of at least 20, at least 50, at least 100 or at least 200 or more consecutive nucleotides) of a polynucleotide is obtained.

The terms “next-generation sequencing” or “high-throughput sequencing.” as used herein, generally refer to the parallelized sequencing-by-synthesis or sequencing-by-ligation platforms. Non-limiting examples of next-generation sequencing methods include nanopore sequencing methods, electronic-detection-based methods, or single-molecule fluorescence-based methods.

As used herein, a “polymerase” refers to an enzyme that catalyzes polynucleotide synthesis by addition of nucleotide units to a nucleotide chain using DNA or RNA as a template. The term refers to either a complete enzyme as it occurs in nature, or an isolated, active catalytic domain, or fragment. In certain embodiments, the polymerase can be thermostable. A “thermostable polymerase” is an enzyme that is relatively stable to heat when compared, for example, to nucleotide polymerases from E. coli, and which catalyzes the template-dependent polymerization of nucleoside triphosphates. A “thermostable polymerase” retains enzymatic activity for polymerization and exonuclease activities when subjected to the repeated heating and cooling cycles used in PCR. In certain embodiments, the polymerase can be a “DNA polymerase.” In certain embodiments, the DNA polymerase is a “high-fidelity DNA polymerase.” For example, but without any limitation, the polymerase can be PRIME STAR GXL polymerase I, ADVANTAGE HD Polymerase, Q5® High-Fidelity DNA Polymerase, PHUSION® High-Fidelity DNA Polymerase, PLATINUM® Taq DNA Polymerase High Fidelity, KAPA HiFi DNA Polymerase, or KOD DNA Polymerase.

As used herein, the term “reverse transcriptase” refers to an enzyme that catalyzes the formation of DNA from an RNA template. In certain embodiments, reverse transcriptase is a DNA polymerase that can be used for first-strand cDNA synthesis from an RNA template. An RNA template can be, without any limitation, a messenger RNA (mRNA), a microRNA (miRNA), a ribosomal RNA (rRNA), a viral RNA, a total RNA, etc. In certain embodiments, for example, and without any limitation, a reverse transcriptase refers to a template-switching reverse transcriptase such as the murine leukemia virus reverse transcriptase.

The terms “adaptor(s),” “adapter(s)” and “tag(s)” may be used synonymously. An adaptor or tag can be coupled to a polynucleotide sequence to be “tagged” by any approach, including ligation, hybridization, or other approaches.

The term “barcode,” “barcode sequence,” “NeoID” or “molecular barcode,” as used herein, generally refers to a label, or identifier, that conveys or is capable of conveying information about a molecule, e.g., nucleic acid molecule or a protein molecule. In certain embodiments, a barcode can be part of a molecule, e.g., a unique nucleotide configuration or sequence that is contained in a larger nucleic acid sequence. In certain embodiments, a barcode can be a tag attached to a molecule (such as a larger protein). In certain embodiments, a barcode can be unique. In certain non-limiting embodiments, barcodes can include polynucleotide barcodes; random nucleic acid and/or amino acid sequences; and synthetic nucleic acid and/or amino acid sequences. In certain non-limiting embodiments, a barcode can be added to a fragment of a deoxyribonucleic acid (DNA) or ribonucleic acid (RNA) sample before, during, and/or after sequencing of the sample. Barcodes can allow for identification and/or quantification of individual sequencing-reads that, if attached to a corresponding molecule, represent the identification of that molecule by the sequence of the barcode.

As used herein, the term “unique molecular identifier” refers to a type of molecular barcoding that provides error correction and increased accuracy during sequencing. Using UMIs reduces the rate of false-positive variant calls and increases sensitivity of variant detection. In silico analysis of the UMI can provide results with a high level of accuracy and report unique reads, removing potential errors. Further details regarding UMI can be found in Islam et al., Nature methods 11.2 (2014): 163, herein incorporated by reference in its entirety.

As used herein, the term “template-switching oligonucleotide” or “TSO” (also referred to as a “template switch oligonucleotide”) refers to an oligonucleotide template to which a polymerase switches from an initial template (e.g., a template mRNA as described herein) during a nucleic acid polymerization reaction. A TSO may include one or more nucleotides (or analogs thereof) that are modified or otherwise non-naturally occurring. For example, without any limitation, the template-switching oligonucleotide can include one or more nucleotide analogs (e.g., LNA, FANA, 2′-0-methyl ribonucleotides, 2′-fluoro ribonucleotides, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the template-switching oligonucleotide.

The term “T cell receptor” or “TCR”, as used herein, refers to a polypeptide expressed on the membrane surface of CD4+ and CD8+ T lymphocytes. TCRβ are antigen receptors that function as a component of the immune system for recognition of peptides bound to self major histocompatibility complex (MHC) molecules on the surface of antigen-presenting cells. The TCR may be a heterodimer of two disulfide-linked transmembrane polypeptide chains, α and β, or γ and δ. Each of these four TCR polypeptide chains is encoded by a distinct genetic locus containing multiple discontinuous gene segments. These include variable (V) region gene segments, joining (J) region gene segments and constant (C) region gene segments. Beta and delta chains contain an additional element termed the diversity (D) gene segment. The variable region contributes to the determination of the particular antigen and MHC molecule to which the TCR has binding specificity. The term TCR, as used herein, includes each of the four polypeptide chains individually, as well as biologically active fragments thereof, including fragments soluble in aqueous solutions, of either chain alone or both chains joined. Biologically active fragments may maintain the ability to bind with specificity to a specific antigen.

As used herein, “NeoTCR” refers to an exogenous T cell receptor (TCR) that is introduced into a T cell, e.g., by gene-editing methods.

“T Cell Product,” as used herein, refers to a composition comprising one or more T cells comprising an exogenous TCR. In certain embodiments, T Cell Products include autologous precision genome-engineered CD8⁺ and/or CD4⁺ T cells. Using a targeted DNA-mediated non-viral precision genome engineering approach, expression of the endogenous TCR is eliminated and replaced by a patient-specific exogenous TCR isolated from peripheral CD8⁺ T cells targeting the tumor-exclusive antigens. In certain embodiments, the resulting engineered CD8⁺ or CD4⁺ T cells express an exogenous TCR on their surface of native sequence, native expression levels, and native TCR function. The sequences of the exogenous TCR external binding domain and cytoplasmic signaling domains are unmodified from the TCR isolated from native CD8⁺ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states.

The term “endogenous” as used herein refers to a nucleic acid molecule or polypeptide that is normally expressed in a cell or tissue.

The term “exogenous” as used herein refers to a nucleic acid molecule or polypeptide that is not endogenously present in a cell. The term “exogenous” would therefore encompass any recombinant nucleic acid molecules or polypeptides expressed in a cell, such as foreign, heterologous, and over-expressed nucleic acid molecules and polypeptides. By “exogenous” nucleic acid is meant a nucleic acid that is not present in a native wild-type cell; for example, an exogenous nucleic acid may vary from an endogenous counterpart by sequence, position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

As used herein, the term “antigen peptide” refers to a peptide (e.g., 9-mer, 10-mer, etc.) that is bound or able to bind into the binding groove of either MHC class 1 or MHC class 2.

As used herein, the term “peptide/MHC complex” refers to a functional molecule comprising an MI-IC protein and an antigen peptide. In certain embodiments, the MHC protein can be an MHC Class 1 protein. In certain embodiments, the peptide/M-IC complex comprises an MI-IC protein, a beta-2 microglobulin, and an antigen peptide. In certain embodiments, the MHC protein can be an MHC Class II protein. 2. METHODS OF PREPARING cDNA

The present disclosure provides compositions and methods for improving the preparation of cDNAs comprising full-length TCR sequences and their use in determining the full-length nucleic acid sequence of those TCRs.

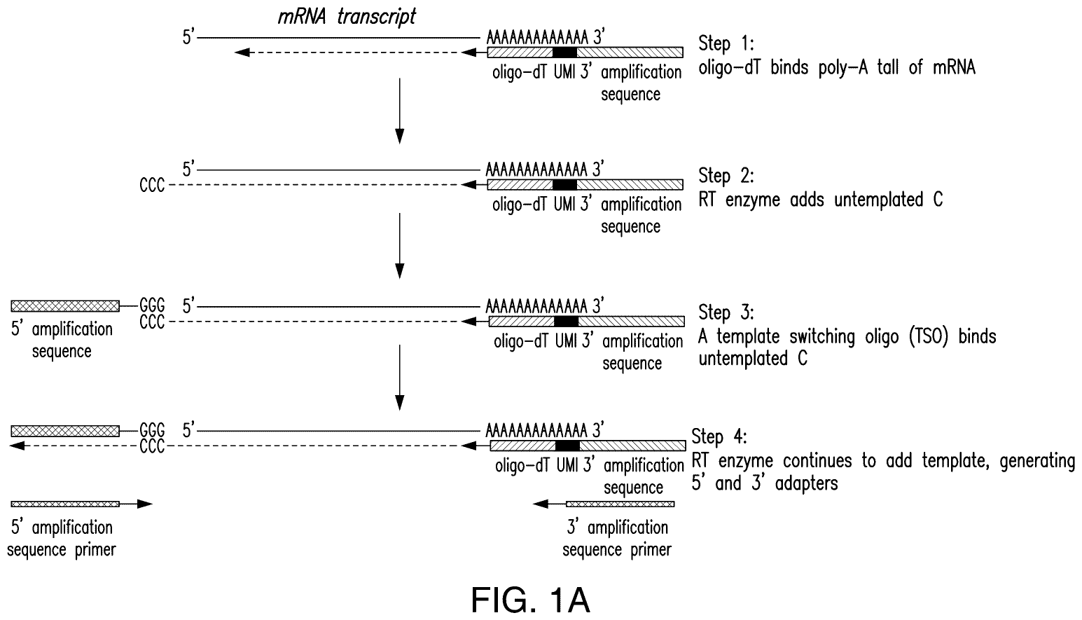

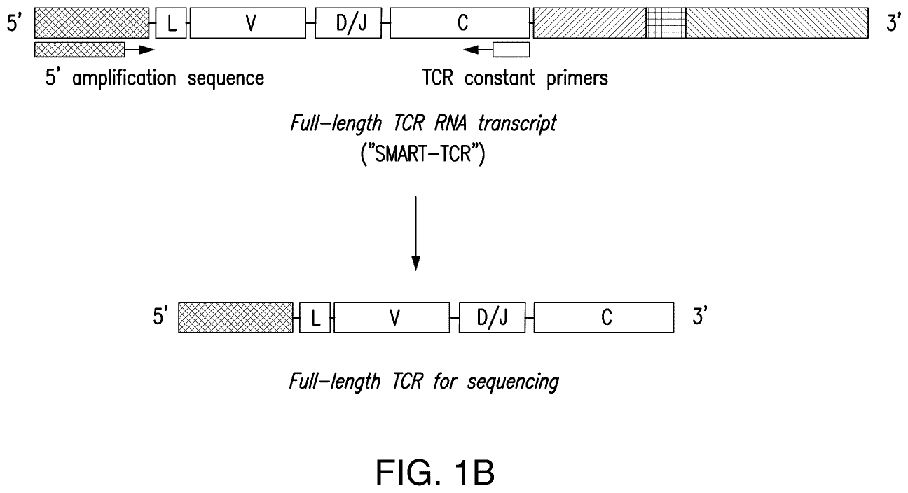

FIGS. 1A and 1B illustrate certain embodiments of the presently disclosed subject matter. For example, in certain embodiments, the mRNA transcripts identified in FIG. 1A are initially extracted from a sample, e.g., a single T cell, comprising TCR expressing cells. Such populations of mRNA transcripts will include full-length TCR sequences. In certain embodiments, the mRNA transcripts are combined with a cDNA synthesis primer and a reverse transcriptase to produce a cDNA-RNA intermediate as illustrated in Step 2. While the cDNA synthesis primer can comprise any sequence present downstream, i.e., towards the 3′ end of the mRNA transcript, relative to the coding sequence of the TCR, in certain embodiments, the cDNA synthesis primer is an oligo-dT oligonucleotide. In certain embodiments, the oligo-dT oligonucleotide comprises a polynucleotide having a sequence set forth in SEQ ID NOs: 1 or 2. SEQ ID NOs: 1 and 2 are provided below:

[SEQ ID NO: 1]
AAGCAGTGGTATCAACGCAGAGTACTTTTTTTTTTTTTTTTTTTTTTTT
TTTTTTVN.
[SEQ ID NO: 2]
AAGCAGTGGTATCAACGCAGAGTACNNNNNNNNTTTTTTTTTTTTTTTT
TTTTTTTTTTTTTTVN

In certain embodiments, a second reverse transcriptase reaction is conducted by combining the cDNA-RNA intermediate with a TSO to produce a cDNA comprising the full-length TCR sequence. In certain embodiments, and as illustrated in Step 3 of FIG. 1A, the TSO can hybridize to a non-templated stretch of cytosine nucleotides incorporated at the 3′ end of the cDNA molecule by the reverse transcriptase. Such hybridization of the TSO to the cDNA provides a template for further extension by a reverse transcriptase, thereby incorporating the complement of the TSO into the further extended cDNA molecule as illustrated in Step 4 of FIG. 1A.

In certain embodiments, the cDNA synthesis primer and/or the TSO can include an amplification primer site. In certain embodiments, the cDNA synthesis primer and/or the TSO can include an identification tag, e.g., a barcode. In certain embodiments, the cDNA synthesis primer and/or the TSO can include a unique molecular identifier (UMI). In certain embodiments, the TSO comprises a polynucleotide having a sequence set forth in any one of SEQ ID NOs: 3-5. SEQ ID NOs: 3-5 are provided below.

[SEQ ID NO: 3]
AGGCAGTGGTATCAACGCAGAGTACATrGrG+G
[SEQ ID NO: 4]
/5Biosg/AAGCAGTGGTATCAACGCAGAGTACATrGrG+G
[SEQ ID NO: 5]
AGAGACAGAAGCAGTGGTATCAACGCAGAGTACATNNNNNNNNrGrG+G

In certain embodiments, the reverse transcriptase has template-switching activity. In certain embodiments, the reverse transcriptase can add a few non-templated nucleotides after it reaches the 5′ end of the template (e.g., RNA). In certain embodiments, the reverse transcriptase is a wild type Moloney Murine Leukemia Virus or a variant thereof. For example, without any limitation, the reverse transcriptase can be a SUPERSCRIPT II®, POWERSCRIPT®, or a SMARTSCRIPT®.

As shown in FIG. 1B, after the reverse transcription, the cDNA can be combined with a first set of primers and a DNA polymerase to obtain a full-length TCR for sequencing.

In certain embodiments, the amplification of the cDNA outlined in FIG. 1B can comprise multiple rounds of PCR. In certain embodiments, distinct sets of amplification primers can be employed. In certain embodiments, the distinct sets of amplification primers can hybridize to distinct portions of the cDNA molecule or amplification products derived therefrom and/or comprise distinct sequences, e.g., UMIs or other types of barcodes and/or amplification sequences.

For example, but not by way of limitation, certain embodiments of the present disclosure will comprise a first PCR reaction (PCR1) that produces a first PCR product which includes the full-length TCR sequence. The first set of primers can include a forward primer annealing to an outer amplification primer site of the TSO, e.g., outer primer, and a reverse primer annealing to a TCR constant sequence. In certain embodiments, the outer primer anneals to an outer amplification binding site of the TSO. In certain embodiments, the outer primer anneals to the TSO. In certain embodiments, the outer primer anneals to a region that is upstream of the TCR variable region. For example, but without any limitation, the outer primer anneals to the TCR leader sequence, designed as “L” in FIG. 1B. In certain embodiments, the outer primer comprises or consists of a polynucleotide having a sequence set forth in any one of SEQ ID NO: 6.

In certain embodiments, the reverse primer anneals to a TRAC sequence or a TRBC sequence.

In certain embodiments, the reverse primer anneals to a conserved sequence of the TCRα sequences.

In certain embodiments, the reverse primer anneals to a conserved sequence of the TCRβ sequences.

In certain embodiments, the reverse primer anneals to a conserved sequence of the TCRα and TCRβ sequences. For example, but without any limitation, the reverse primer anneals to both TRBC1 and TRBC2 genes. In certain embodiments, the reverse primer anneals to a region that is downstream of the TRAC sequence. In certain embodiments, the reverse primer anneals to a region that is downstream of the TRBC sequence. In certain embodiments, the reverse primer anneals to a region that is downstream of the V-A-C joining region of the TCRα sequence. For example, without any limitation, the reverse primer anneals to a region that is about 200 bp downstream of the V-J-C joining region of the TCRα sequence. In certain embodiments, the reverse primer anneals to a region that is downstream of the V-D-J-C joining region of the TCRβ sequence. For example, without any limitation, the reverse primer anneals to a region that is about 200 bp downstream of the V-D-J-C joining region of the TCRβ sequence. In certain embodiments, the reverse primer comprises or consists of a polynucleotide having a sequence set forth in any one of SEQ ID NOs: 7 or 8. SEQ ID NOs: 6-8 are provided below:

[SEQ ID NO: 6]
AAGCAGTGGTATCAACGCAGAGT
[SEQ ID NO: 7]
GCCACAGCACTGTTGCTCTTGAAGTCC
[SEQ ID NO: 8]
CCACCAGCTCAGCTCCACGTG

In certain embodiments, the first PCR reaction (PCR1) further produces a first PCR product which includes the NeoID sequence. The first set of primers can include forward and reverse primers annealing to regions flanking the NeoID sequence. In certain embodiments, the primer comprises or consists of a polynucleotide having a sequence set forth in SEQ ID NO: 9 and 10. SEQ ID NO: 9 and 10 are provided below:

[SEQ ID NO: 9]
CTCGCCACGTCGGCTATCCTG
[SEQ ID NO: 10]
GAGGTCGCTGTAGCTTGCTCACG

In certain embodiments, once the first PCR product is obtained, second and third PCR reactions (PCR2 and PCR3, respectively) can be conducted. In certain embodiments, the second PCR comprises amplifying the TCRα, TCRβ, and/or NeoID sequences. In certain embodiments, the second PCR comprises combining the first PCR product with a second set of primers to produce a second PCR product. The second set of primers includes a forward primer annealing to an inner amplification primer site of the TSO, e.g., inner primer, and a reverse primer annealing to a TCR constant sequence. In certain embodiments, the inner primer anneals to a TSO adapter sequence. In certain embodiments, the inner primer anneals to the TSO. In certain embodiments, the inner primer anneals to a region downstream of the TSO and upstream of the TCR variable region. For example, but without any limitation, the inner primer anneals to the TCR leader sequence. In certain embodiments, the inner primer comprises an adapter sequence. In certain non-limiting embodiments, an adapter sequence is a polynucleotide non-homologous to the TCR template. In certain embodiments, the adapter sequence can be a binding site for further amplification, e.g., PCR3. In certain embodiments, the inner primer comprises or consists of a polynucleotide having a sequence set forth in SEQ ID NO: 11. In certain embodiments, the reverse primer anneals to a TRAC sequence or a TRBC sequence. In certain embodiments, the reverse primer anneals to a conserved sequence of the TCRα sequences. In certain embodiments, the reverse primer anneals to a conserved sequence of the TCRβ sequences. In certain embodiments, the reverse primer anneals to a conserved sequence of the TCRα and TCRβ sequences. In certain embodiments, the reverse primer anneals to a region that is downstream of the TRAC sequence. In certain embodiments, the reverse primer anneals to a region that is downstream of the TRBC sequence. In certain embodiments, the reverse primer anneals to a region that is downstream of the V-J-C joining region of the TCRα sequence. In certain embodiments, the reverse primer anneals to a region that is downstream of the V-D-J-C joining region of the TCRβ sequence. In certain embodiments, the reserve primer anneals to a region that is upstream of the binding region of the reverse primer used in the PCR1. In certain embodiments, the reverse primer comprises an adapter sequence. In certain non-limiting embodiments, the adapter sequence is a polynucleotide non-homologous to the TCR template. In certain embodiments, the adapter sequence can be a binding site for further amplification, e.g., PCR3. In certain embodiments, the reverse primer comprises or consists of a polynucleotide having a sequence set forth in any one of SEQ ID NOs: 7-8. In certain embodiments, the reverse primer comprises or consists of a polynucleotide having a sequence set forth in SEQ ID NOs. 12-13. SEQ ID NO: 11-13 are provided below:

[SEQ ID NO: 11]
ACTCGCGAGGGACGTGAAGCN₍₄₋₃₀₎AAGCAGTGGTATCAACGCAGAGT
[SEQ ID NO: 12]
GACAAAACTGTGCTAGACATGAGG
[SEQ ID NO: 13]
CAGGGAAGAAGCCTGTGGCCAGG

In certain embodiments, the second PCR reaction (PCR2) further produces a second PCR product which includes the NeoID sequence. The second set of primers can include forward and reverse primers nested on a NeoID template. In certain embodiments, the primers include an adapter sequence. In certain embodiments, the adapter sequence can be a binding site for further amplification, e.g., PCR3. In certain embodiments, the primer comprises or consists of a polynucleotide having a sequence set forth in SEQ ID NO: 14 and 15. SEQ ID NO: 14 and 15 are provided below:

[SEQ ID NO: 14]
CCAGGGTTTTCCCAGTCACGACN₍₄₋₃₀₎CACGTCGGCTATCCTGATCGGATGAAGCAGTGGTATCAACGCAGAGT
[SEQ ID NO: 15]
AGCGGATAACAATTTCACACAGGAN₍₄₋₃₀₎CGCTGTAGCTTGCTCACGTCCAG

In certain embodiments, the third PCR reaction (PCR3) comprises amplifying the TCRα, TCRβ, and/or NeoID sequences. In certain embodiments, the third PCR comprises combining the second PCR product with a third set of primers to produce a third PCR product. The third set of primers includes a forward primer. In certain embodiments, the forward primer anneals to the TSO adapter sequence. In certain embodiments, the forward primer anneals to the TSO. In certain embodiments, the forward primer anneals to a region that is downstream of the TSO. For example, without any limitation, the forward primer anneals to the TCR leader sequence. In certain embodiments, the forward primer anneals to an adapter sequence of an inner primer. For example, without any limitation, the forward primers anneal to the adapter sequence of the inner primer used in the second TCR reaction. In certain embodiments, the forward primer comprises an adapter sequence in 5′. In certain embodiments, the adapter sequence in 5′ is compatible with a DNA sequencing system. Non-limiting examples of adapter sequences compatible with a DNA sequencing system include P7 adapter. In certain embodiments, the forward primer comprises a barcode. Non-limiting examples of a barcode include i7 barcode. In certain embodiments, the forward primer anneals to an adapter are added during the second PCR amplification of the NeoID sequences. Additional information regarding the forward primer for the amplification of NeoID sequence can be found in the Example section. In certain embodiments, the forward primer comprises or consists of a polynucleotide having a sequence set forth in SEQ ID NOs: 18 and 19. The third set of primers also includes a reverse primer. In certain embodiments, the reverse primer anneals to a TRAC sequence or a TRBC sequence. In certain embodiments, the reverse primer anneals to a conserved sequence of the TCRα sequences. In certain embodiments, the reverse primer anneals to a conserved sequence of the TCRβ sequences. In certain embodiments, the reverse primer anneals to a conserved sequence of the TCRα and TCRβ sequences. In certain embodiments, the reverse primer anneals to a region that is downstream of the TRAC sequence. In certain embodiments, the reverse primer anneals to a region that is downstream of the TRBC sequence. In certain embodiments, the reverse primer anneals to a region that is downstream of the V-J-C joining region of the TCRα sequence. In certain embodiments, the reverse primer anneals to a region that is downstream of the V-D-J-C joining region of the TCRβ sequence. In certain embodiments, the reserve primer anneals to a region that is upstream of the binding region of the reverse primer used in the PCR1. In certain embodiments, the reserve primer anneals to a region that is upstream of the binding region of the reverse primer used in the PCR2. In certain embodiments, the reverse primer comprises an adapter sequence in 5′. In certain embodiments, the adapter sequence in 5′ is compatible with a DNA sequencing system. Non-limiting examples of adapter sequences compatible with a DNA sequencing system include P5 adapter. In certain embodiments, the forward primer comprises a barcode. Non-limiting examples of a barcode include i5 barcode. In certain embodiments, the reverse primer anneals to an adapter are added during the second PCR amplification of the NeoID sequences. Additional information regarding the reverse primer for the amplification of NeoID sequence can be found in the Example section. In certain embodiments, the reverse primer comprises or consists of a polynucleotide having a sequence set forth in SEQ ID NOs: 16, 17, and 20.

In certain embodiments, fourth and fifth PCR reactions (PCR4 and PCR5, respectively) can be performed using the third and the fourth PCR products, respectively. The fourth PCR can be performed by combining the third PCR product with a fourth set of primers to add adapter sequences, which may thereby improve sequencing output, or sequencing efficiency, including high-throughput sequencing (although such adaptor sequences can be incorporated at any of the preceding steps). In certain embodiments, the fifth PCR can be performed by combining the fourth PCR product with a fourth set of primers to add adapter sequences, thereby improving sequencing reading, including high-throughput sequencing (although, again, such adaptors can be added at any of the preceding steps). The fourth set of primers can include a forward primer annealing to the 5′ sequence of the second and third PCR products and a reverse primer annealing to a TCR constant sequence of the second and third PCR products.

In certain embodiments, the DNA polymerase in one or more of the amplification steps described herein is at a final concentration from about 0.001 units/μl to about 10 units/μl. In certain embodiments, the DNA polymerase is at a final concentration from about 0.001 units/μl to about 1 units/μl. In certain embodiments, the DNA polymerase is at a final concentration from about 0.001 units/μl to about 0.1 units/μl. In certain embodiments, the DNA polymerase is at a final concentration from about 0.01 units/μl to about 1 units/μl. In certain embodiments, the DNA polymerase is at a final concentration from about 0.01 units/μl to about 0.1 units/μl. In certain embodiments, the DNA polymerase is at a final concentration from about 0.02 units/μl to about 0.08 units/μl. In certain embodiments, the DNA polymerase in one or more of the amplification steps described herein can be added to the reaction. For example, but without any limitation, additional DNA polymerase can be supplemented into the reactions if enzyme activity and/or TCR recovery appear to be low.

In certain embodiments, the method comprises a first PCR reaction (PCR1) comprising a DNA polymerase at a final concentration of about 0.02 units/pd. In certain embodiments, the method comprises a second PCR reaction (PCR2) comprising a DNA polymerase at a final concentration of about 0.04 units/μl. In certain embodiments, the method comprises a third PCR reaction (PCR3) comprising a DNA polymerase at a final concentration of about 0.04 units/μl. In certain embodiments, the method comprises a fourth PCR reaction (PCR4) comprising a DNA polymerase at a final concentration of about 0.04 units/μl. In certain embodiments, the method comprises a fifth PCR reaction (PCR5) comprising a DNA polymerase at a final concentration of about 0.04 units/μl.

In certain embodiments, the first, second, third, fourth, and fifth PCR reactions comprise an extension step. In certain embodiments, the extension step of the first PCR (PCR1) has a time of about 90 seconds to about 6 minutes. In certain embodiments, the extension step of the second PCR (PCR2) has a time from about 20 seconds to about 1 minute. In certain embodiments, the extension step of the third PCR (PCR3) has a time from about 20 seconds to about 1 minute. In certain embodiments, the extension step of the fourth PCR (PCR4) has a time from about 20 seconds to about 1 minute. In certain embodiments, the extension step of the fifth PCR (PCR5) has a time from about 20 seconds to about 1 minute.

In certain embodiments, the primers of any of the reactions disclosed herein can have a concentration from about 0.01 μM to about 1 μM. In certain embodiments, the method comprises a first PCR (PCR1) that has a forward primer at a concentration from about 0.01 μM to about 1 μM. In certain embodiments, the method comprises a first PCR (PCR1) that has a reverse primer at a concentration from about 0.01 μM to about 1 μM. In certain embodiments, the method comprises a second PCR (PCR2) that has a forward primer at a concentration from about 0.01 μM to about 1 μM. In certain embodiments, the method comprises a second PCR (PCR2) that has a reverse primer at a concentration from about 0.01 μM to about 1 μM. In certain embodiments, the method comprises a third PCR (PCR3) that has a forward primer at a concentration from about 0.01 μM to about 1 μM. In certain embodiments, the method comprises a third PCR (PCR3) that has a forward primer at a concentration of about 0.1 μM. In certain embodiments, the method comprises a third PCR (PCR3) that has a reverse primer at a concentration from about 0.01 μM to about 1 μM. In certain embodiments, the method comprises a third PCR (PCR3) that has a forward primer at a concentration from about 0.3 μM. In certain embodiments, the method comprises a fourth PCR (PCR4) that has a forward primer at a concentration from about 0.01 μM to about 1 μM. In certain embodiments, the method comprises a fourth PCR (PCR4) that has a reverse primer at a concentration from about 0.01 μM to about 1 μM. In certain embodiments, the method comprises a fifth PCR (PCR5) that has a forward primer at a concentration from about 0.01 μM to about 1 μM. In certain embodiments, the method comprises a fifth PCR (PCR5) that has a forward primer at a concentration of about 0.1 μM. In certain embodiments, the method comprises a fifth PCR (PCR5) that has a reverse primer at a concentration from about 0.01 μM to about 1 μM. In certain embodiments, the method comprises a fifth PCR (PCR5) that has a forward primer at a concentration of about 0.3 μM.

In certain embodiments, the method comprises a product of the third, fourth, and fifth PCR can be sequenced by next-generation sequencing methods. Non-limiting examples of sequencing platforms that can be used within the presently disclosed subject matter include Roche GS 20, Roche JS FLX, the Solexa platform, the Supported Oligonucleotide Ligation and Detection (SOLiD) platform, the HeliScope platform, and the Oxford Nanopore Technologies platform. In certain embodiments, the method comprises obtaining reads for the TCRα sequence and the TCRβ sequence.

### 3. Methods of Analyzing T Cells

The present disclosure provides compositions and methods for analyzing single cells, e.g., T cells. The understanding of T cell and TCR repertoire can be useful for improving personalized and tailored therapies, e.g., adoptive cell transfer. In certain embodiments, the present disclosure includes sorting of T cells from a sample. In certain embodiments, the sample comprising T cells is collected from a subject. The sample can be any sample of bodily fluid or tissue containing T cells. Non-limiting examples of fluid or tissue include blood, thymus, spleen, lymph nodes, bone marrow, a tumor biopsy, or an inflammatory lesion biopsy. In certain embodiments, the sample can be collected from a pathological site. For example, but without any limitation, the sample can be collected from tumor tissue. In certain embodiments, the sample can include cells collected from a subject and after growth in vitro cell culture.

In certain embodiments, the T cells are isolated from the sample and sorted. In certain embodiments, the T cells can be sorted into separate locations in a container. For example, but without any limitation, the T cells can be sorted in a multi-well plate (e.g., 384-well plate) or microwell array, capillaries or tubes (e.g., 0.2 mL tubes), or chambers in a microfluidic device. In certain embodiments, the T cells can be sorted in emulsion droplets that spatially separate cells. For example, but without any limitation, T cells can be sorted using a flow cytometer. In certain embodiments, the T cells can be labeled before being sorted. In certain embodiments, the T cells can be labeled with an antibody (e.g., anti-CD3 antibody) that specifically binds a T cell protein. In certain embodiments, the T cells can be labeled with multiple reagents to classify a T cell subtype (e.g., a CD8 T cell, a CD4 T cell, etc.). In certain embodiments, a subset of T cells within a sample can be sorted as single cells into separate locations (e.g., separate emulsion droplets). For example, a subset of CD3⁺/CD8⁺ can be sorted for performing the methods disclosed herein. In certain embodiments, T cells are sorted as described in International PCT Application Nos. PCT/US2020/017887, PCT/US2019/025415, and PCT/US2020/054732, each of which is incorporated by reference herein.

In certain embodiments, the T cells are sorted using a peptide/MHC complex. In certain embodiments, the peptide/MHC complex comprises an antigen peptide, a β2-microglobulin, and an MHC class 1 heavy chain. In certain embodiments, the peptide/MHC complex is labeled with a barcode or NeoID. In certain embodiments, the peptide/MHC complex is a comPACT polypeptide, as described in International Patent Publication No. WO 2019195310, the content of which is incorporated by reference in its entirety.

In certain embodiments, the T cells are lysed to liberate the molecules, such as RNAs. Non-limiting examples of methods to lyse the T cells include osmotic shock, thermal lysis, mechanical lysis, chemical lysis, or optical lysis.

In certain embodiments, the present disclosure includes methods for isolating nucleic acid molecules, e.g., RNA molecules, from the sample. In certain non-limiting embodiments, isolation of nucleic acid molecules can be performed by phenol/chloroform extraction, ethanol precipitation, electrophoresis, and/or chromatography.

In certain embodiments, the presently disclosed methods for analyzing single T cells include any of the methods described herein in Section 2.

In certain embodiments, the presently disclosed methods for analyzing single T cells include methods for whole transcriptome analysis of the T cells. “Whole transcriptome analysis,” as used herein, refers to the evaluation of all or a fraction of the transcriptome of a sample, e.g., a single T cell. In certain embodiments, whole transcriptome analysis includes amplification of the transcripts using various PCR or non-PCR-based methods. For example, the transcripts, e.g., mRNA, micro-RNA, siRNA, tRNA, rRNA, and any combination thereof, can be amplified using one or more universal primers binding a plurality of RNAs, such as mRNA molecules.

In certain embodiments, the present disclosure provides methods for analyzing single T cells that are specific for an antigen peptide. In certain embodiments of the methods disclosed herein, the T cells are initially sorted using a peptide/MHC complex comprising a NeoID. In certain embodiments, the T cells are analyzed by any of the methods described herein in Section 2. In certain embodiments, the presently disclosed methods for analyzing single T cells include sequencing the TCRα sequence, the TCRβ sequence, and the NeoID. In certain embodiments, the presently disclosed methods further comprise the removal of false-positive T cells (e.g., T cells not specific for the antigen peptide). For example, but without any limitation, false-positive T cells can be removed by sequence analysis of the NeoID bound to the sorted T cell. The presence of multiple copies of the same NeoID yields a high ratio of specific NeoID barcode species compared to non-specific bound NeoID. This will result in a higher signal-to-noise NeoID ratio (e.g., S/N ratio). In certain embodiments, the S/N ratio is above a threshold. In certain embodiments, the threshold is about 10. In certain embodiments, non-specific T cells will bind relatively equal numbers of different peptide/MHC complexes resulting in a lower ratio of distinct NeoID. In certain embodiments, non-specific T cells will have an S/N ratio below a threshold (e.g., below a threshold of about 10).

In certain embodiments, sorted T cells can recognize two different peptide/MHC complexes. In these instances, the calculated signal-to-noise NeoID ratio can be below a threshold (e.g., below a threshold of about 10). In certain embodiments, a first signal-to-noise NeoID ratio (S/N1) and a second signal-to-noise NeoID ratio (S/N2) can be calculated.

In certain embodiments, S/N1 is the highest signal divided by the second-highest signal. In certain embodiments, S/N2 is the highest signal from one peptide/MHC complex divided by the highest signal from a different peptide/MHC complex. In certain embodiments, the highest signal from a different peptide/MHC complex is not the second highest signal in the sample.

### 4. Compositions and Kits

The present disclosure also provides compositions for performing the methods disclosed herein. In certain embodiments, the present disclosure includes enzymes, primers, buffers, salts and other components. For example, without any limitation, the compositions can include one or more controls (e.g., positive or negative control), a reverse transcriptase, a TSO, dNTPs, buffers and co-factors (e.g., a salt, a metal cofactor, etc.), one or more enzyme-stabilizing components (e.g., DTT), and any other desired reaction mixture component(s). In certain embodiments, the compositions can be present in one or more reaction tubes.

The present disclosure also provides kits for performing the methods disclosed herein. In certain embodiments, the kit can be used to prepare a cDNA comprising a full-length TCR sequence. In certain non-limiting embodiments, the kits disclosed herein can include a cDNA synthesis primer, e.g., an oligo-dT, a reverse transcriptase, dNTPs, buffers and co-factors, primers, a TSO, one or more enzyme-stabilizing components (e.g., DTT), and/or any other reagents for performing the sequencing. In certain embodiments, the kits include reagents for isolating RNA from a nucleic acid source.

Components of the subject kits may be present in separate containers, or multiple components may be present in a single container. For example, without any limitation, a cDNA synthesis primer and a reverse transcriptase buffer may be provided in separate containers or may be provided in a single container. In certain embodiments, one or more kit components is provided in a lyophilized form such that the components are ready to use and may be conveniently stored at room temperature.

In addition to the above-mentioned components, a subject kit may further include instructions for using the components of the kit. e.g., to practice the subject method. The instructions for practicing the subject method are generally recorded on a suitable recording medium. For example, the instructions may be printed on a substrate, such as paper or plastic, etc. For example, without any limitation, the instructions can be present in the kits as a package insert, in the labeling of the container of the kit or components thereof (i.e., associated with the packaging or subpackaging), etc. In certain embodiments, the instructions are available from a remote source, e.g. download from a website.

### 5. T Cell Products

In certain embodiments, using the gene-editing technology and TCR isolation technology described in PCT/US2020/17887 and PCT/US2019/025415, which are incorporated herein in their entireties, the TCR identified by the methods disclosed herein (see Sections 2 and 3 above) can be cloned in autologous CD8 and CD4⁺ T cells from the same subject with cancer by precision genome engineered to express an exogenous TCR (e.g., a NeoTCR). In other words, the TCRβ identified by the methods disclosed herein, which are tumor-specific and identified in cancer patients, are inserted into the cancer patient's T cells. T cells expressing TCRβ identified by the methods disclosed herein are then expanded in a manner that preserves a “young” T cell phenotypes, in which the majority of the T cells exhibit T memory stem cell and T central memory phenotypes.

These ‘young’ or ‘younger’ or less-differentiated T cell phenotypes are described to confer improved engraftment potential and prolonged persistence post-infusion. Thus, the administration of these T cell products, comprising ‘young’ T cells, has the potential to benefit patients with cancer, through improved engraftment potential, prolonged persistence post-infusion, and rapid differentiation into effector T cells to eradicate tumor cells throughout the body.

In certain embodiments, the manufacturing process of these T cells products (e.g., including exogenous TCRβ identified by the methods disclosed herein) involves electroporation of dual ribonucleoprotein species of CRISPR-Cas9 nucleases bound to guide RNA sequences, with each species targeting the genomic TCRα locus and the genomic TCRβ locus. The comprehensive assessment of the T cell product and precision genome engineering process indicates that the NeoTCR Product will be well tolerated following infusion back to the patient.

The genome engineering approach described herein enables the highly efficient generation of bespoke T cells expressing TCRβ identified by the methods disclosed herein for personalized adoptive cell therapy for patients with solid and liquid tumors. Furthermore, the engineering method is not restricted to the use in T cells and has also been applied successfully to other primary cell types, including natural killer and hematopoietic stem cells. Additional information on the methods used to generate T cell products using the TCRβ identified by the methods disclosed herein can be found in International Patent Publication No. WO 2019/089610.

### 6. Exemplary Embodiments

A1. In certain non-limiting embodiments, the present disclosure provides a method of preparing a deoxyribonucleic acid (DNA) comprising a full-length T cell receptor (TCR) sequence, the method comprising: a) obtaining a TCR complementary DNA (cDNA) sequence by combining a ribonucleic acid (RNA) molecule with a cDNA synthesis primer complementary to a region of the RNA molecule 3′ to the TCR coding sequence, a template-switching oligonucleotide (TSO), and a reverse transcriptase under conditions sufficient for the reverse transcriptase to produce the cDNA sequence; and b) preparing a DNA comprising a full-length TCR sequence by combining the cDNA with a first set of amplification primers comprising an outer primer annealing to the TSO sequence and a primer annealing to a TCR constant sequence and a polymerase under conditions sufficient for the polymerase to produce a DNA comprising a full-length TCR sequence.
A2. The foregoing method of A1, further comprising sequencing the DNA comprising a full-length TCR sequence.
A3. The foregoing method of A1, wherein the RNA molecule is extracted from a sample comprising T cells.
A4. The foregoing method of A3, wherein the sample is collected from a subject.
A5. The foregoing method of any one of A1-A4, wherein the cDNA synthesis primer is an oligo-dT primer.
A6. The foregoing method of A5, wherein the oligo-dT primer comprises a polynucleotide having a sequence set forth in SEQ ID NOs: 1 or 2.
A7. The foregoing method of A1-A6, wherein the TSO comprises an amplification primer site, a barcode, and a unique molecular identifier (UMI).
A8. The foregoing method of A1-A7, wherein the TSO comprises a polynucleotide having a sequence set forth in any one of SEQ ID NOs: 3-5.
A9. The foregoing method of A1-A8, wherein the reverse transcriptase is a template-switching reverse transcriptase.
A10. The foregoing method of any one of A1-A9, wherein the outer primer comprises a polynucleotide having a sequence set forth in SEQ ID NOS: 6.
A11. The foregoing method of any one of A1-A10, wherein the inner primer comprises a polynucleotide having the sequence set forth in SEQ ID NO: 11.
A12. The foregoing method of any one of A1-A11, wherein the primer annealing to a TCR constant sequence comprises a polynucleotide having the sequence set forth in SEQ ID NOS: 7, 8, 12, and 13.
A13. The foregoing method of anyone of A1-A12, wherein the DNA comprising a full-length TCR sequence is further amplified using a second set of amplification primers, wherein one or both of the primers comprise a barcode sequence.
A14. The foregoing method of any one of A1-A13, wherein the full-length TCR receptor comprises a TCRα chain and/or a TCRβ chain.
A15. The foregoing method of any one of A1-A13, wherein the full-length TCR receptor comprises a TCR gamma chain and/or a TCR delta chain.
A16. The foregoing method of any one of A1-A15, wherein b) comprises an extension phase from about 20 seconds to about 90 seconds.
A17. The foregoing method of any one of A1-A16, wherein the amplification primers comprises a forward primer at a concentration from about 0.1 μM to about 0.6 μM.
B1. In certain non-limiting embodiments, the present disclosure provides a cDNA library produced by the foregoing method of any one of A1-A17.
C1. In certain non-limiting embodiments, the present disclosure provides a method of analyzing single T cells to determine the nucleic acid sequence of a full-length TCR sequence, comprising: a) sorting single T cells from a sample comprising a plurality of T cells; b) preparing a DNA comprising a full-length TCR sequence; and c) sequencing the DNA comprising a full-length TCR sequence.
C2. The foregoing method of C1, wherein preparing a DNA comprising a full-length TCR sequence comprises: i) obtaining a TCR complementary DNA (cDNA) sequence by combining a ribonucleic acid (RNA) molecule with a cDNA synthesis primer complementary to a region of the RNA molecule 3′ to the TCR coding sequence, a template-switching oligonucleotide (TSO), and a reverse transcriptase under conditions sufficient for the reverse transcriptase to produce the cDNA sequence; and ii) preparing a DNA comprising a full-length TCR sequence by combining the cDNA with a first set of amplification primers comprising an outer primer annealing to the TSO sequence and a primer annealing to a TCR constant sequence and a polymerase under conditions sufficient for the polymerase to produce a DNA comprising a full-length TCR sequence.
C3. The foregoing method of C1 or C2, wherein the sample is collected from a subject.
C4. The foregoing method of any one of C1-C3, wherein the cDNA synthesis primer is an oligo-dT primer.
C5. The foregoing method of C4, wherein the oligo-dT primer comprises a polynucleotide having a sequence set forth in SEQ ID NOs: 1 or 2.
C6. The foregoing method of any one of C1-C5, wherein the TSO comprises an amplification primer site, an identification tag, and a unique molecular identifier (UMI).
C7. The foregoing method of any one of C1-C6, wherein the TSO comprises a polynucleotide having a sequence set forth in any one of SEQ ID NOs: 3-5.
C8. The foregoing method of any one of C1-C7, wherein the reverse transcriptase is a template-switching reverse transcriptase.
C9. The foregoing method of any one of C1-C8, wherein the outer primer comprises a polynucleotide having the sequence set forth in any one of SEQ ID NO: 6.
C10. The foregoing method of any one of C1-C9, wherein the inner primer comprises a polynucleotide having the sequence set forth in SEQ ID NO: 11.
C11. The foregoing method of any one of C1-C10, wherein the primer annealing to a TCR constant sequence comprises a polynucleotide having the sequence set forth in any one of SEQ ID Nos. 7, 8, 12, and 13.
C12. The foregoing method of any one of C1-C11, wherein the amplification primers comprise a barcode sequence.
C13. The foregoing method of any one of C1-C12, wherein the full-length TCR receptor comprises a TCRα chain and/or a TCRβ chain.
C14. The foregoing method of any one of C1-C12, wherein the full-length TCR receptor comprises a TCRγ chain and/or a TCRδ chain.
C15. The foregoing method of any one of C1-C14, wherein b) comprises an extension phase from about 20 seconds to about 90 seconds.
C16. The foregoing method of any one of C1-C15, wherein the amplification primers comprises a forward primer at a concentration from about 0.1 μM to about 0.6 μM.
C17. The foregoing method of any one of C1-C16, further comprising analyzing the whole transcriptome of the single T cells.
C18. The foregoing method of any one of C1-C17, further comprising analyzing somatic mutation or genetic polymorphisms of the single T cells.
C19. The foregoing method of any one of C1-C18, wherein the sorting comprises contacting the sample with of a plurality of peptide/MHC complexes, wherein each peptide/MHC complex comprises an associated barcode.
C20. The foregoing method of C19, further comprising sequencing the barcode associated with each peptide/MHC complex.
C21. The foregoing method of C20, further comprising determining a ratio of a first barcode associated with a first peptide/MHC complex and a second barcode associated with a second peptide/MHC complex.
C22. The foregoing method of C21, further comprising determining antigen specificity of the T cell based on the ratio of the first barcode and the second barcode.

### EXAMPLES

The presently disclosed subject matter will be better understood by reference to the following Examples, which are provided as exemplary of the presently disclosed subject matter, and not by way of limitation.

### Example 1

Traditional methods for cloning of TCR are based on a combination of reverse transcription PCR followed by a first and second round of nested PCR reactions which amplify the VJ segment of the TCRα chain and the VDJ segment of the TCRβ chain. These segments are then analyzed in silico to allow the reconstruction of the full-length TCR. However, these methods are of limited use. As indicated in the table below, approximately 12% of the TCR cloning shows ambiguous results. This ambiguity could be related to the features of the primers that can mask certain variations during the amplification steps. Thus, it is important to overcome this limitation.

| TCR Classification | Problem | Consensus TCR | Percentage |
|---|---|---|---|
| Clear | None | One | 82% |
| Ambiguous | Silent mutation | One | 3% |
| Ambiguous | Truncation | One | 3% |
| Ambiguous | TRBV7-9 | Two | 10% |
| Ambiguous | Other Issues | Two | 2% |

### Materials and Methods:

### Cell Sorting.

Cells were sorted based on T cell phenotype and binding to a peptide, MHC complex. Cells were sorted into a 96-well plate containing lysis buffer described in the table below. Additional information regarding methods for cell sorting can be found in International Patent Application No. PCT/US20/17887, the content of which is incorporated by reference in its entirety.

| Reagent | Final Concentration | |
|---|---|---|
| Triton X-100 | 0.5% | |
| RNAse inhibitor | 0.8 | units/μL |
| oligo-dT | 2 | uM |
| dNTP | 2 | mM |

Nuclease-free water

SMART-TCR, Step 1: reverse transcription (RT).

Before beginning the RT reaction, the mixture containing the cells and sorting reagents (as described in the section “Cell Sorting” above) was placed on a thermocycler with a heated lid and incubated as detailed in the table below:

| Temperature (° C.) | Time (min.) | Cycle |
|---|---|---|
| 72 | 3 | 1 |

For each RT reaction, the following components were added to a single PCR reaction:

| Reagent | Final Concentration | |
|---|---|---|
| First-strand buffer | 0.96 X | |
| Betaine | 0.96 | M |
| Magnesium Chloride | 5.76 | mM |
| DTT | 4.8 | mM |
| sP238 primer [SEQ ID NO: 4] | 0.96 | μM |
| SMARTScribe Enzyme | 9.13 | U/μl |
| RNase inhibitor | 0.96 | U/μL |

Nuclease-free water

Each reaction was incubated in a thermocycler with a heated lid as detailed in the table below:

| Temperature (° C.) | Time (min.) | Cycle |
|---|---|---|
| 42 | 90 | 1 |
| 50 | 2 | 2-9 |
| 42 | 2 | |
| 70 | 15 | 10 |
| 4 | Hold | 11 |

SMART-TCR, Step 2: PCR amplification 1. For each reaction, a first PCR amplification was performed using a 5′ amplification primer annealing on the TSO-sequence and a 3′ amplification primer annealing on the constant region of the TCR. In the first PCR (PCR1), each reaction was prepared as follows:

| Reagent | Final Concentration | |
|---|---|---|
| KAPA HiFi Mix | 1 X | |
| sP239 | 0.1 | μM |
| a0001 primer | 0.6 | μM |
| a0003 primer | 0.6 | μM |
| S0009 primer | 0.8 | μM |
| a0017 primer | 0.8 | μM |

Nuclease-free water

S0009 and a0017 primers were designed to target flanking (one 5′ and one 3′) sequences of the NeoID. a0001 and a0003 primers were designed to bind to the TCR constant region (a001 binds to the TCRα constant region, and a0003 binds to the TCRβ constant region). sP239 was designed to bind to the TSO sequence introduced in the RT step (sP238). The primer sequences are provided below:

sP239:
[SEQ ID NO: 6]
AAGCAGTGGTATCAACGCAGAGT
a0001:
[SEQ ID NO: 7]
GCCACAGCACTGTTGCTCTTGAAGTCC
a0003:
[SEQ ID NO: 8]
CCACCAGCTCAGCTCCACGTG
S0009:
[SEQ ID NO: 9]
CTCGCCACGTCGGCTATCCTG
a0017:
[SEQ ID NO: 10]
GAGGTCGCTGTAGCTTGCTCACG

Each reaction was incubated in a thermocycler with a heated lid as detailed in the table below:

| | | |
|---|---|---|
| Temperature (° C.) | Time (m/s) | Cycle |

| | | | |
|---|---|---|---|
| 98 | 3 | m | 1 |
| 98 | 20 | s | 2-30 |
| 64 | 15 | s | |
| 72 | 90 | s | |
| 72 | 5 | m | 31 |

| | | |
|---|---|---|
| 4 | Hold | 32 |

SMART-TCR, Step 3: PCR Amplification 2.

A second PCR was performed to further amplify the PCR1 product by using a nested 5′ amplification sequence primer and a 3′ primer annealing on the constant region of the TCR. In the second PCR, different conditions and primers were used whether the target is a TCRα or TCRβ sequence or a NeoID sequence. For TCRα amplification, each reaction was prepared as follows:

| Reagent | Final Concentration | |
|---|---|---|
| KOD Master Mix | 1 X | |
| sP259 | 0.6 | μM |
| TRAC_Primer2 primer | 0.3 | μM |
| PCR1 product | 1.5-2 | μl |

Nuclease-free water

sP259 primer was designed to anneal to the TSO sequence from the PCR1 step (sP239) and to add a small adapter sequence to the 5′ end (indicated by the N in SEQ ID NO: 11). The adapter sequence will serve as a binding site for forward primers during the third PCR amplification step. TRAC_Primer2 binds to the TCRα constant region, slightly 5′ (nested inward) to the a0001 primer used in the PCR1 step. The primer sequences are provided below:

sP259:
[SEQ ID NO: 11]
ACTCGCGAGGGACGTGAAGCN₍₄₋₃₀₎AAGCAGTGGTATCAACGCAGAGT
TRAC_Primer2:
[SEQ ID NO: 12]
GACAAAACTGTGCTAGACATGAGG

For TCRβ amplification, each reaction was prepared as follows:

| Reagent | Final Concentration | |
|---|---|---|
| KOD Master Mix | 1 X | |
| sP259 | 0.6 | μM |
| aP0042 primer | 0.3 | μM |
| PCR1 product | 1.5-2 | μl |

Nuclease-free water

aP0042 primer was designed to bind to the TCRβ constant region, slightly 5′ (nested inward) to the a0003 primer used in the PCR1 step. The primer sequence is provided below:

aP0042:
[SEQ ID NO: 13]
CAGGGAAGAAGCCTGTGGCCAGG

For TCRα and TCRβ amplification, each reaction was incubated in a thermocycler with a heated lid as detailed in the table below:

| | | |
|---|---|---|
| Temperature (° C.) | Time (m/s) | Cycle |

| | | | |
|---|---|---|---|
| 95 | 3 | m | 1 |
| 95 | 20 | s | |
| 60 | 30 | s | 2-20 |
| 72 | 1 | m | |
| 72 | 10 | m | 21 |

| | | |
|---|---|---|
| 12 | Hold | 22 |

For NeoID amplification, each reaction was prepared as follows:

| Reagent | Final Concentration | |
|---|---|---|
| KOD Master Mix | 1 X | |
| S0010 primer | 0.1 | μM |
| aP0043 primer | 0.1 | μM |
| PCR1 product | 1.5-2 | μl |

Nuclease-free water

S0010 was designed to anneal to a slightly 3′ (nested) region on the NeoID template from the original binding site of S0009 in the first PCR step. aP0043 was designed to bind to a slightly 5′ (nested) region on the NeoID template from the original binding site of a0017 in the first PCR step. Both S0010 and aP0043 included a small adapter sequence to the 5′ end (for S0010) or the 3′ end (for aP0043) that will serve as primer binding sites during the 3^{rd} PCR amplification. The primer sequences are provided below:

S0010:
[SEQ ID NO: 14]
CCAGGGTTTTCCCAGTCACGACN₍₄₋₃₀₎CACGTCGGCTATCCTGATCGGATGAAGCAGTGGTATCAACGCAGAGT
aP0043:
[SEQ ID NO: 15]
AGCGGATAACAATTTCACACAGGAN₍₄₋₃₀₎CGCTGTAGCTTGCTCACGTCCAG

For NeoID amplification, each reaction was incubated in a thermocycler with a heated lid as detailed in the table below:

| | | |
|---|---|---|
| Temperature (° C.) | Time (m/s) | Cycle |

| | | | |
|---|---|---|---|
| 95 | 2 | m | 1 |
| 95 | 20 | s | 2-20 |
| 60 | 10 | s | |
| 70 | 20 | s | |

| | | |
|---|---|---|
| 12 | Hold | 21 |

### SMART-TCR. Step 4: PCR Amplification 3—Sequencing Adapter Addition.

Sequencing adapters and indexing primers were added to the amplified products of the PCR amplification 2. For adapter addition to PCR products obtained in PCR amplification 2 (PCR2 product), a reaction was prepared as follows:

| Reagent | Final Concentration | |
|---|---|---|
| KOD Master Mix | 1 X | |
| PCR-3 barcode primers | 0.3 | μM |
| PCR2 product (from Reaction 3-5) | 1.5-2 | μl |

Nuclease-free water

PCR3 barcode primers for TCRα and TCRβ included a mixture of 2 kinds of primers. Forward primers were designed to bind the adapter sequence added during the second PCR amplification (sP259) and to add a well-specific barcode (i7 barcode) as well as an Illumina sequencing-ready P7 sequence. Reverse primers were designed to bind a nested-in TCR constant region (a sequence 5′ from the TRAC_Primer2 sequence or the aP0042 sequence) and to add a well-specific barcode (i5 barcode) as well as an Illumina sequencing-ready P5 sequence.

For NeoID primers, the forward primers were designed to bind the 5′ adapter sequence added during the second PCR amplification (S0010) and the reverse primers bind the 3′ adapter sequence added during the second PCR amplification (aP0043). The NeoID forward primers included a well-specific barcode (i7 barcode) as well as an Illumina sequencing-ready P7 sequence. The NeoID reverse primers included a well-specific barcode (i5 barcode) as well as an Illumina sequencing-ready P5 sequence. The general structure of the primers used in the third round PCR primers is provided below:

Reverse primer for TCRα constant region:
[SEQ ID NO: 16]
AATGATACGGCGACCACCGAGATCTACAC[i5
barcode]TCGTCGGCAGCGTCAGATGTGTATAAGAGACAGN₍₄₋₃₀₎ACGGCAGGGTCAGGGTTCTG
Reverse primer for TCRβ constant region:
[SEQ ID NO: 17]
AATGATACGGCGACCACCGAGATCTACAC[i5
barcode]TCGTCGGCAGCGTCAGATGTGTATAAGAGACAGN₍₄₋₃₀₎AGCGACCTCGGGTGGGAAC
Forward primer for TCRα and TCRβ:
[SEQ ID NO: 18]
CAAGCAGAAGACGGCATACGAGAT[i7
barcode]GTCTCGTGGGCTCGGAGATGTGTATAAGAGACAGN₍₄₋₃₀₎ACTCGCGAGGGACGTGAAGC
Forward primer for NeoID:
[SEQ ID NO: 19]
CAAGCAGAAGACGGCATACGAGAT[i7
barcode]GTCTCGTGGGCTCGGAGATGTGTATAAGAGACAGN₍₄₋₃₀₎CCAGGGTTTTCCCAGTCACGAC
Reverse primer for NeoID:
[SEQ ID NO: 20]
AATGATACGGCGACCACCGAGATCTACAC[i5
barcode]TCGTCGGCAGCGTCAGATGTGTATAAGAGACAGN₍₄₋₃₀₎AGCGGATAACAATTTCACACAGGA

For TCRα and TCRβ amplification (PCR3), each reaction was incubated in a thermocycler with a heated lid as detailed in the table below:

| | | |
|---|---|---|
| Temperature (° C.) | Time (m/s) | Cycle |

| | | | |
|---|---|---|---|
| 95 | 2 | m | 1 |
| 95 | 20 | s | |
| 60 | 10 | s | 2-20 |
| 70 | 1 | m | |
| 70 | 5 | m | 21 |

| | | |
|---|---|---|
| 12 | Hold | 22 |

For NeoID amplification (PCR3), each reaction was incubated in a thermocycler with a heated lid as detailed in the table below:

| | | |
|---|---|---|
| Temperature (° C.) | Time (m/s) | Cycle |

| | | | |
|---|---|---|---|
| 95 | 2 | m | 1 |
| 95 | 20 | s | 2-20 |
| 60 | 10 | s | |
| 70 | 20 | s | |

| | | |
|---|---|---|
| 12 | Hold | 21 |

### SMART-TCR. Step 5: DNA Purification.

PCR products from PCR amplification 3 were pooled and separated by agarose electrophoresis. The TCRα and TCRβ sequences show a molecular weight of approximately 650-750 bp, while the NeoID showed a molecular weight of 200 bp. The identified bands were cut and processed for purification. The purified DNA was quantified and concentrated for further processing.

### SMART-TCR. Step 6: Sequencing and Data Analysis.

The purified and concentrated DNA was then further processed for library preparation and sequencing using the EQ-005 Illumina Mini-Seq®system. Reads for TCRα, TCRβ, and NeoID were analyzed in silico with a custom computational pipeline. Briefly, to identify TCR candidates, a cell must contain a TCRα and a TCRβ chain. A NeoID signal to noise (S/N) analysis is then conducted and only cells with TCRα and TCRβchains having a NeoID S/N ratio higher than or equal to 10 are selected. NeoID refers to a unique oligonucleotide sequence labeling a unique pHLA (peptide-HLA) tetramer. The ratio of reads for that unique pHLA tetramer, divided by the reads identified for a different pHLA tetramer, gives the S/N ratio, a way to estimate the specificity of a TCR for a pHLA.

### Results:

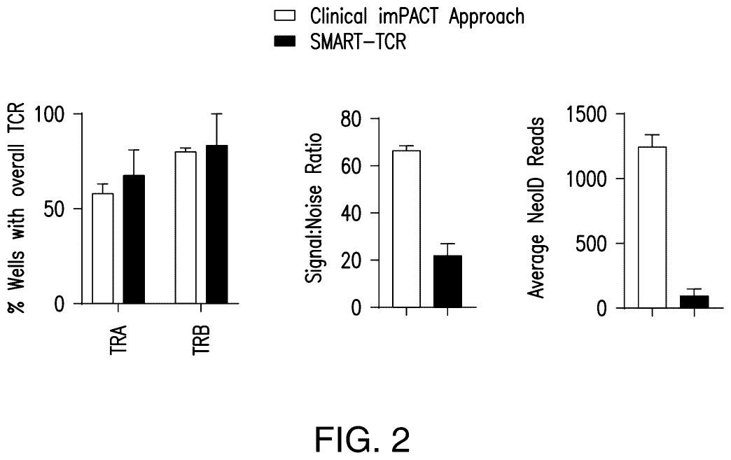

To determine the performance features of the SMART-TCR protocol disclosed herein, a comparison between the traditional nested PCR approach (hereinafter “Clinical imPACT approach”) and the SMART-TCR protocol was performed. As illustrated in FIG. 2, the SMART-TCR protocol showed a higher TCR recovery rate, a lower S/N ratio and a reduced NeoID read count compared to the imPACT protocol.

To improve the S/N ratio and the NeoID read count, several steps of the SMART-TCR protocol were modified according to the table below:

| SMART-TCR | Modification |
|---|---|
| Step 1 | Clean up of RT-PCR product |
| Step 2 | Increase amount of enzyme (enzyme spike-in) |
| Step 3 | Increase amount of NeoID primer |
| Step 4 | Decrease extension time |

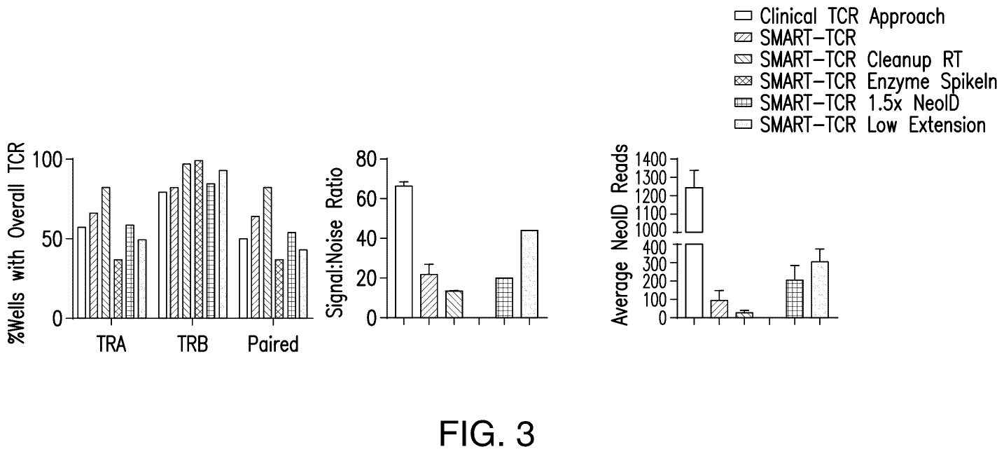

As shown in FIG. 3, increasing the amount of NeoID primers and decreasing the extension time improved the S/N ratio and the NeoID read count as compared to the SMART-TCR protocol, while clean up and increased enzyme amount did not substantially increase S/N ratio or NeoID read count.

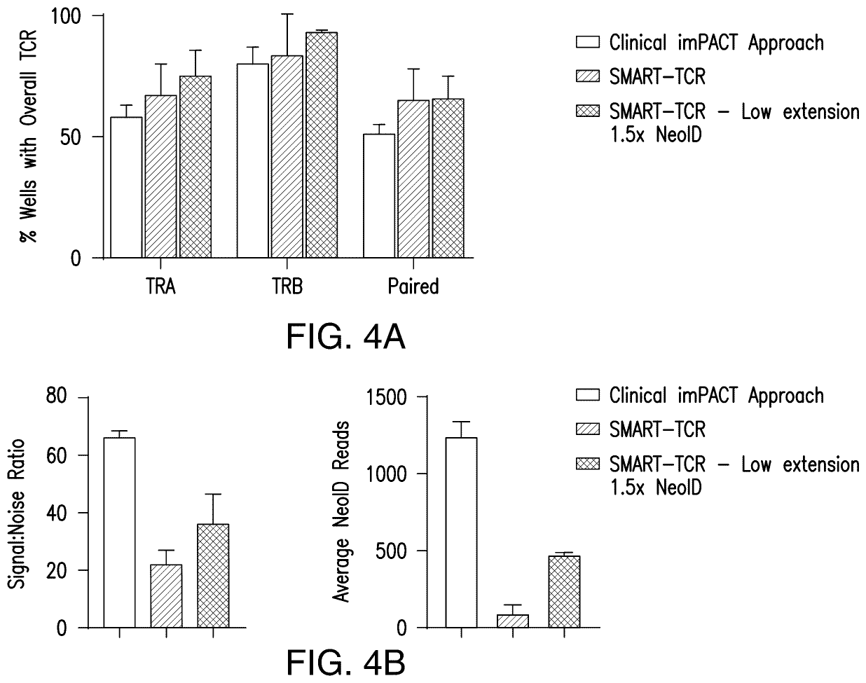

Next, it was determined whether lowering the extension time and increasing the concentration of NeoID primers together would yield better recovery of TCR sequences. Without being bound by any particular theory, lowering the extension time may cut out other competing transcripts that require longer extension times. Surprisingly, and shown in FIGS. 4A and 4B, this approach yielded a better TCR recovery rate (FIG. 4A) and improved significantly the S/N ratio and NeoID read counts as compared to the SMART-TCR protocol at the conventional extension time and NeoID primer concentration (FIG. 4B).

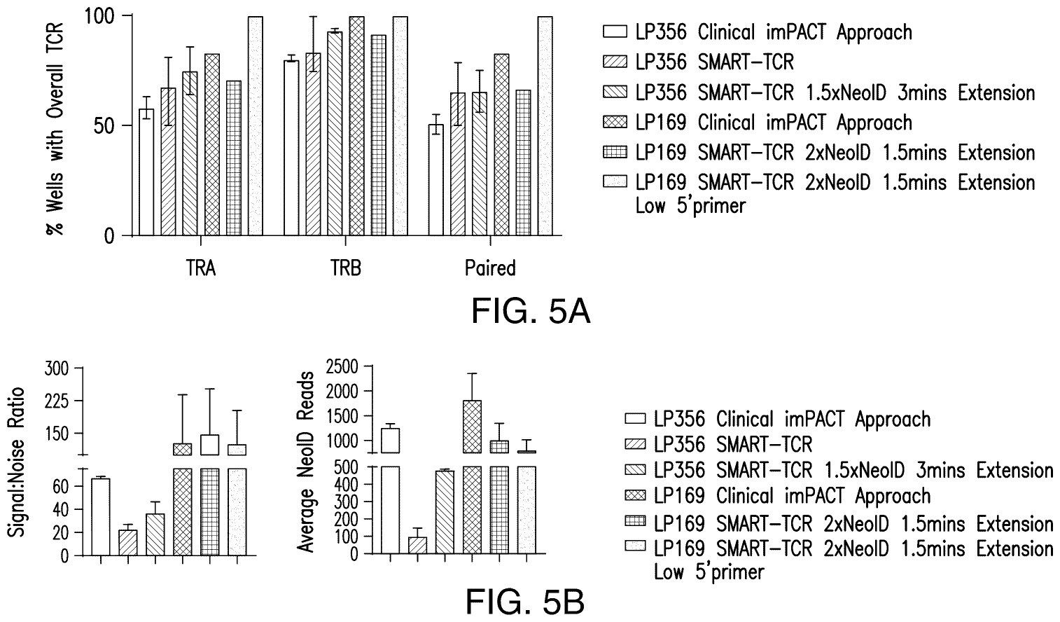

This approach was replicated in primary cells obtained from human subjects (LP356 and LP169). Further to the increased amount of NeoID primers and decreased extension time, the SMART-TCR protocol was modified by reducing the amount of 5′ primer in Step 2(PCR1 As shown in FIGS. 5A and 5B, this new approach increased significantly the TCR recovery rate (FIG. 5A), the S/N ratio and the NeoID read count (FIG. 5B).

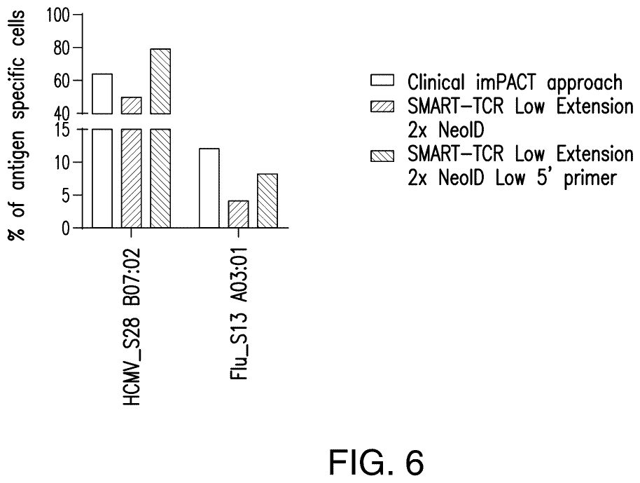

Finally, it was confirmed that the SMART-TCR protocol yielded comparable HLA-matched comPACT distribution compared to the clinical imPACT approach (FIG. 6). Overall, these data demonstrate that the unexpected and superior qualities of the SMART-TCR protocol, including variations involving reduced extension time, increased NeoID primer concentration, and/or reduced 5′ primer concentration. As described herein, this method can provide high-quality and unbiased sequences of T cell receptors regardless of whether the sequences are in databases or not. Further, this method allows the identification of antigen specificity from the same sample and can be easily adapted to any personalized medicine pipeline.

### Example 2

Alternative primers can be used in the methods disclosed herein and in Example 1. In a first option (Option A), part of the DNA sequences for the TSO (sP238) and the oligo-dT are the same (e.g., SEQ ID NOs: 1-5). Alternatively (Option B), the DNA sequences for the TSO and the oligo-dT are different.

In Option B, the use of a different oligo-dT sequence reduces the off-binding effects of the forward primer (sP239) in Step 2 and the forward primer (sP259) in Step 3. In Option B, sP238, sP239, and sP259 are used simultaneously with swapping out any of the oligo-dT sequences below. In such a scenario, the following sequences are used:

S-oligo-dT:
[SEQ ID NO: 21]
ACGAGCATCAGCAGCATACGATTTTTTTTTTTTTTTTTTTTTTTTTTTTTTVN
W-oligo-dT:
[SEQ ID NO: 22]
ATTCTAGAGGCCGAGGCGGCCGACATGNNNNNNNNTTTTTTTTTTTTTTTTTTTTTTTTTTTTTTVN
N-oligo-dT:
[SEQ ID NO: 23]
ACTATCTAGAGCGGCCGCNNNNNNNNTTTTTTTTTTTTTTTTTTTTTTTTTTTTTTVN
B-oligo-dT:
[SEQ ID NO: 24]
ACACTCTTTCCCTACACGACGCTCTTCCGATCTNNNNNNNNTTTTTTTTTTTTTTTTTTTTTTTTTTTTTTVN
TTTVN
K-oligo-dT:
[SEQ ID NO: 25]
TAGAGGCCGAGGCGGCCGACATGNNNNNNNNTTTTTTTTTTTTTTTTTTTTTTTTTTTTTTVN

Alternative TSO and forward primer sequences are used interchangeably with any of the oligo-dT sequences above, so long as the main sequence of the TSO, the forward primer for Step 2, and the forward primer for Step 3 overlap to some degree. TSO and forward primers are grouped by letters that correspond to the letter printed above for the oligo-dT sequences. The TSO and forward primers in each reaction are from the same letter and share some degree of overlap in DNA sequence:

S-TSO:
[SEQ ID NO: 26]
/5BiosG/AGAGACAGATTGCGCAATGNNNNNNNNrGrGrG
S3-PCR1-forward primer:
[SEQ ID NO: 27]
AGAGACAGATTGCGCAATG
S3-PCR2-forward primer:
[SEQ ID NO: 28]
ACTCGCGAGGGACGTGAAGC AGAGACAGATTGCGCAATG
W-TSO:
[SEQ ID NO: 29]
/5BiosG/ATTCTAGAGGCCGAGGCGGCCGACATGrGrGrG
W-PCR1-forward primer:
[SEQ ID NO: 30]
ATTCTAGAGGCCGAGGCGGCCGACATG
W-PCR2-forward primer:
[SEQ ID NO: 31]
ACTCGCGAGGGACGTGAAGCN₍₄₋₃₀₎ATTCTAGAGGCCGAGGCGGCCGACATG
N-TSO:
[SEQ ID NO: 32]
/5BiosG/ACTATCTAGAGCGGCCGC/GrGrG
N-PCR1-forward primer:
[SEQ ID NO: 33]
ACTATCTAGAGCGGCCGC
N-PCR2-forward primer:
[SEQ ID NO: 34]
ACTCGCGAGGGACGTGAAGCN₍₄₋₃₀₎ACTATCTAGAGCGGCCGC
B-TSO:
[SEQ ID NO: 35]
/5BiosG/ACACTCTTTCCCTACACGACGCTCTTCCGATCTrGrGrG
B-PCR1-forward primer:
[SEQ ID NO: 36]
ACACTCTTTCCCTACACGACGCTCTTCCGATCT
B-PCR2-forward primer:
[SEQ ID NO: 37]
ACTCGCGAGGGACGTGAAGCACACTCTTTCCCTACACGACGCTCTTCCGATCT
K-TSO:
[SEQ ID NO: 38]
/5BiosG/TAGAGGCCGAGGCGGCCGACATGrGrGrG
K-PCR1-forward primer:
[SEQ ID NO: 39]
TAGAGGCCGAGGCGGCCGACATG
K-PCR2-forward primer:
[SEQ ID NO: 40]
ACTCGCGAGGGACGTGAAGCTAGAGGCCGAGGCGGCCGACATG

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of preparing a deoxyribonucleic acid (DNA) comprising a full-length T cell receptor (TCR) sequence, the method comprising:
a) obtaining a TCR complementary DNA (cDNA) sequence by combining a ribonucleic acid (RNA) molecule with a cDNA synthesis primer complementary to a region of the RNA molecule 3′ to the TCR coding sequence, a template-switching oligonucleotide (TSO), and a reverse transcriptase under conditions sufficient for the reverse transcriptase to produce the cDNA sequence; and
b) preparing a DNA comprising a full-length TCR sequence by combining the cDNA with a first set of amplification primers comprising an outer primer annealing to the TSO sequence and a primer annealing to a TCR constant sequence and a polymerase under conditions sufficient for the polymerase to produce a DNA comprising a full-length TCR sequence.

2. The method of claim 1, further comprising sequencing the DNA comprising a full-length TCR sequence.

3-4. (canceled)

5. The method of claim 1, wherein the cDNA synthesis primer is an oligo-dT primer.

6. (canceled)

7. The method of claim 1, wherein the TSO comprises an amplification primer site, a barcode, and a unique molecular identifier (UMI).

8. The method of claim 1, wherein the TSO comprises a polynucleotide having a sequence set forth in any one of SEQ ID NOs: 3-5.

9. The method of claim 1, wherein;
a) the reverse transcriptase is a template-switching reverse transcriptase;
b) the outer primer comprises a polynucleotide having a sequence set forth in SEQ ID NOS: 6;
c) the inner primer comprises a polynucleotide having the sequence set forth in SEQ ID NO: I11;
d) the primer annealing to a TCR constant sequence comprises a polynucleotide having the sequence set forth in SEQ ID NOS: 7, 8, 12, and 13; or
e) the DNA comprising a full-length TCR sequence is further amplified using a second set of amplification primers, wherein one or both of the primers comprise a barcode sequence.

10-13. (canceled)

14. The method of claim 1, wherein the full-length TCR receptor comprises a TCRα chain and/or a TCRβ chain, or a TCR gamma chain and/or a TCR delta chain.

15. (canceled)

16. The method of claim 1, wherein b) comprises an extension phase from about 20 seconds to about 90 seconds, or wherein the amplification primers comprises a forward primer at a concentration from about 0.1 μM to about 0.6 μM.

17. (canceled)

18. A cDNA library produced by the method of claim 1.

19. A method of analyzing single T cells to determine the nucleic acid sequence of a full-length TCR sequence, comprising:
a) sorting single T cells from a sample comprising a plurality of T cells;
b) preparing a DNA comprising a full-length TCR sequence, comprising:
i. obtaining a TCR complementary DNA (cDNA) sequence by combining a ribonucleic acid (RNA) molecule with a cDNA synthesis primer complementary to a region of the RNA molecule 3′ to the TCR coding sequence, a template-switching oligonucleotide (TSO), and a reverse transcriptase under conditions sufficient for the reverse transcriptase to produce the cDNA sequence; and
ii. preparing a DNA comprising a full-length TCR sequence by combining the cDNA with a first set of amplification primers comprising an outer primer annealing to the TSO sequence and a primer annealing to a TCR constant sequence and a polymerase under conditions sufficient for the polymerase to produce a DNA comprising a full-length TCR sequence; and
c) sequencing the DNA comprising a full-length TCR sequence.

20. (canceled)

21. The method of claim 19, wherein the cDNA synthesis primer is an oligo-dT primer.

22. (canceled)

23. The method of claim 19, wherein the TSO comprises an amplification primer site, an identification tag, and a unique molecular identifier (UMI).

24. (canceled)

25. The method of claim 19, wherein the reverse transcriptase is a template-switching reverse transcriptase.

26. The method of claim 19, wherein;
a) the outer primer comprises a polynucleotide having the sequence set forth in any one of SEQ ID NO: 6;
b) the inner primer comprises a polynucleotide having the sequence set forth in SEQ ID NO: 11;
c) the primer annealing to a TCR constant sequence comprises a polynucleotide having the sequence set forth in any one of SEQ ID Nos. 7, 8, 12, and 13; or
d) the amplification primers comprise a barcode sequence.

27-29. (canceled)

30. The method of claim 19, wherein the full-length TCR receptor comprises a TCRα chain and/or a TCRβ chain, or a TCRγ chain and/or a TCRδ chain.

31. (canceled)

32. The method of claim 19, wherein b) comprises an extension phase from about 20 seconds to about 90 seconds.

33. The method of claim 19, wherein the amplification primers comprises a forward primer at a concentration from about 0.1 μM to about 0.6 μM.

34. The method of claim 19, further comprising analyzing the whole transcriptome of the single T cells.

35. (canceled)

36. The method of claim 19, wherein the sorting comprises contacting the sample with of a plurality of peptide/MHC complexes, wherein each peptide/MHC complex comprises an associated barcode.

37. The method of claim 36, further comprising:
a) sequencing the barcode associated with each peptide/MHC complex;
b) determining a ratio of a first barcode associated with a first peptide/MHC complex and a second barcode associated with a second peptide/MHC complex; or
c) determining antigen specificity of the T cell based on the ratio of the first barcode and the second barcode.

38-39. (canceled)


SEQUENCE LISTING (40 sequences)

SEQ ID NO: 1 - DNA, 57 bp, synthetic construct
  misc_feature 1..57: note = oligo-dT oligonucleotide
  variation 57: note = n is a, c, g, or t
  source 1..57: mol_type = other DNA
aagcagtggtatcaacgcagagtacttttttttttttttttttttttttttttttvn

SEQ ID NO: 2 - DNA, 65 bp, synthetic construct
  misc_feature 1..65: note = oligo-dT oligonucleotide
  variation 26..33: note = n is a, c, g, or t
  variation 65: note = n is a, c, g, or t
  source 1..65: mol_type = other DNA
aagcagtggtatcaacgcagagtacnnnnnnnnttttttttttttttttttttttttttttttvn

SEQ ID NO: 3 - DNA, 30 bp, synthetic construct
  misc_feature 1..30: note = Template-switching oligonucleotides component
  rRNA 28..29: note = rG
  misc_feature 1..27: note = DNA
  misc_feature 28..29: note = RNA
  misc_feature 30: note = DNA
  modified_base 30: mod_base = OTHER; note = locked nucleic acid (LNA) guanylate
  source 1..30: mol_type = other DNA
aagcagtggtatcaacgcagagtacatggg

SEQ ID NO: 4 - DNA, 30 bp, synthetic construct
  misc_feature 1..30: note = Template-switching oligonucleotides component
  modified_base 1: mod_base = OTHER; note = 5'biotin modification, 5Bios
  rRNA 28..29: note = rG
  misc_feature 1..27: note = DNA
  misc_feature 28..29: note = RNA
  misc_feature 30: note = DNA
  modified_base 30: mod_base = OTHER; note = locked nucleic acid (LNA) guanylate
  source 1..30: mol_type = other DNA
ragcagtggtatcaacgcagagtacatggg

SEQ ID NO: 5 - DNA, 46 bp, synthetic construct
  misc_feature 1..46: note = Template-switching oligonucleotides component
  variation 36..43: note = n is a, c, g, or t
  rRNA 44..45: note = rG
  misc_feature 1..43: note = DNA
  misc_feature 44..45: note = RNA
  misc_feature 46: note = DNA
  modified_base 46: mod_base = OTHER; note = locked nucleic acid (LNA) guanylate
  source 1..46: mol_type = other DNA
agagacagaagcagtggtatcaacgcagagtacatnnnnnnnnggg

SEQ ID NO: 6 - DNA, 23 bp, synthetic construct
  misc_feature 1..23: note = Synthetic: sP239
  source 1..23: mol_type = other DNA
aagcagtggtatcaacgcagagt

SEQ ID NO: 7 - DNA, 27 bp, synthetic construct
  misc_feature 1..27: note = Synthetic: a0001
  source 1..27: mol_type = other DNA
gccacagcactgttgctcttgaagtcc

SEQ ID NO: 8 - DNA, 21 bp, synthetic construct
  misc_feature 1..21: note = Synthetic: a0003
  source 1..21: mol_type = other DNA
ccaccagctcagctccacgtg

SEQ ID NO: 9 - DNA, 21 bp, synthetic construct
  misc_feature 1..21: note = Synthetic: S0009
  source 1..21: mol_type = other DNA
ctcgccacgtcggctatcctg

SEQ ID NO: 10 - DNA, 23 bp, synthetic construct
  misc_feature 1..23: note = Synthetic: a0017
  source 1..23: mol_type = other DNA
gaggtcgctgtagcttgctcacg

SEQ ID NO: 11 - DNA, 73 bp, synthetic construct
  misc_feature 1..73: note = Synthetic: sP259
  variation 21..50: note = n at positions 21 - 24 must be present; and n at positions 25-50 can be present or absent
  variation 21..50: note = n, if present, is a, c, g, or t
  variation 25..50: note = position may be absent
  source 1..73: mol_type = other DNA
actcgcgagggacgtgaagcnnnnnnnnnnnnnnnnnnnnnnnnnnnnnnaagcagtggtatcaacgcagagt

SEQ ID NO: 12 - DNA, 24 bp, synthetic construct
  misc_feature 1..24: note = Synthetic: TRAC_Primer2
  source 1..24: mol_type = other DNA
gacaaaactgtgctagacatgagg

SEQ ID NO: 13 - DNA, 23 bp, synthetic construct
  misc_feature 1..23: note = Synthetic: aP0042
  source 1..23: mol_type = other DNA
cagggaagaagcctgtggccagg

SEQ ID NO: 14 - DNA, 99 bp, synthetic construct
  misc_feature 1..99: note = Synthetic: S0010
  variation 23..52: note = n at positions 23 - 26 must be present; and n at positions 27-52 can be present or absent
  variation 23..52: note = n, if present, is a, c, g, or t
  variation 27..52: note = position may be absent
  source 1..99: mol_type = other DNA
ccagggttttcccagtcacgacnnnnnnnnnnnnnnnnnnnnnnnnnnnnnncacgtcggctatcctgatcggatgaagcagtggtatcaacgcagagt

SEQ ID NO: 15 - DNA, 77 bp, synthetic construct
  misc_feature 1..77: note = Synthetic: aP0043
  variation 25..54: note = n at positions 25 - 28 must be present; and n at positions 29-54 can be present or absent
  variation 25..54: note = n, if present, is a, c, g, or t
  variation 29..54: note = position may be absent
  source 1..77: mol_type = other DNA
agcggataacaatttcacacaggannnnnnnnnnnnnnnnnnnnnnnnnnnnnncgctgtagcttgctcacgtccag

SEQ ID NO: 16 - DNA, 112 bp, synthetic construct
  misc_feature 1..112: note = Synthetic: Reverse primer for TCR-alpha constant region
  misc_feature 29^30: note = location of i5 barcode
  variation 63..92: note = n at positions 63 - 66 must be present; and n at positions 67-92 can be present or absent
  variation 63..92: note = n, if present, is a, c, g, or t
  variation 67..92: note = position may be absent
  source 1..112: mol_type = other DNA
aatgatacggcgaccaccgagatctacactcgtcggcagcgtcagatgtgtataagagacagnnnnnnnnnnnnnnnnnnnnnnnnnnnnnnacggcagggtcagggttctg

SEQ ID NO: 17 - DNA, 112 bp, synthetic construct
  misc_feature 1..112: note = Synthetic: Reverse primer for TCR-beta constant region
  misc_feature 29^30: note = location of i5 barcode
  variation 63..92: note = n at positions 63 - 66 must be present; and n at positions 67-92 can be present or absent
  variation 63..92: note = n, if present, is a, c, g, or t
  variation 67..92: note = position may be absent
  variation 93: note = n is a, c, g, or t
  source 1..112: mol_type = other DNA
aatgatacggcgaccaccgagatctacactcgtcggcagcgtcagatgtgtataagagacagnnnnnnnnnnnnnnnnnnnnnnnnnnnnnnnagcgacctcgggtgggaac

SEQ ID NO: 18 - DNA, 108 bp, synthetic construct
  misc_feature 1..108: note = Synthetic: Forward primer for TCR-alpha and TCR-beta
  misc_feature 24^25: note = location of i7 barcode
  variation 59..88: note = n at positions 59 - 62 must be present; and n at positions 63 - 88 can be present or absent
  variation 59..88: note = n, if present, is a, c, g, or t
  variation 63..88: note = position may be absent
  source 1..108: mol_type = other DNA
caagcagaagacggcatacgagatgtctcgtgggctcggagatgtgtataagagacagnnnnnnnnnnnnnnnnnnnnnnnnnnnnnnactcgcgagggacgtgaagc

SEQ ID NO: 19 - DNA, 110 bp, synthetic construct
  misc_feature 1..110: note = Synthetic: Forward primer for NeoID
  misc_feature 24^25: note = location of i7 barcode
  variation 59..88: note = n at positions 59 - 62 must be present; and n at positions 63 - 88 can be present or absent
  variation 59..88: note = n, if present, is a, c, g, or t
  variation 59..62: note = position may be absent
  source 1..110: mol_type = other DNA
caagcagaagacggcatacgagatgtctcgtgggctcggagatgtgtataagagacagnnnnnnnnnnnnnnnnnnnnnnnnnnnnnnccagggttttcccagtcacgac

SEQ ID NO: 20 - DNA, 116 bp, synthetic construct
  misc_feature 1..116: note = Synthetic: Reverse primer for NeoID
  misc_feature 29^30: note = location of i5 barcode
  variation 63..92: note = n at positions 63 - 66 must be present; and n at positions 67-92 can be present or absent
  variation 63..92: note = n, if present, is a, c, g, or t
  variation 67..92: note = position may be absent
  source 1..116: mol_type = other DNA
aatgatacggcgaccaccgagatctacactcgtcggcagcgtcagatgtgtataagagacagnnnnnnnnnnnnnnnnnnnnnnnnnnnnnnagcggataacaatttcacacagga

SEQ ID NO: 21 - DNA, 53 bp, synthetic construct
  misc_feature 1..53: note = Synthetic: S-oligo-dT
  variation 53: note = n is a, c, g, or t
  source 1..53: mol_type = other DNA
acgagcatcagcagcatacgattttttttttttttttttttttttttttttvn

SEQ ID NO: 22 - DNA, 67 bp, synthetic construct
  misc_feature 1..67: note = Synthetic: W-oligo-dT
  variation 28..35: note = n is a, c, g, or t
  variation 67: note = n is a, c, g, or t
  source 1..67: mol_type = other DNA
attctagaggccgaggcggccgacatgnnnnnnnnttttttttttttttttttttttttttttttvn

SEQ ID NO: 23 - DNA, 58 bp, synthetic construct
  misc_feature 1..58: note = Synthetic: N-oligo-dT
  variation 19..26: note = n is a, c, g, or t
  variation 58: note = n is a, c, g, or t
  source 1..58: mol_type = other DNA
actatctagagcggccgcnnnnnnnnttttttttttttttttttttttttttttttvn

SEQ ID NO: 24 - DNA, 73 bp, synthetic construct
  misc_feature 1..73: note = Synthetic: B-oligo-dT
  variation 34..41: note = n is a, c, g, or t
  variation 73: note = n is a, c, g, or t
  source 1..73: mol_type = other DNA
acactctttccctacacgacgctcttccgatctnnnnnnnnttttttttttttttttttttttttttttttvn

SEQ ID NO: 25 - DNA, 63 bp, synthetic construct
  misc_feature 1..63: note = Synthetic: K-oligo-dT
  variation 24..31: note = n is a, c, g, or t
  variation 63: note = n is a, c, g, or t
  source 1..63: mol_type = other DNA
tagaggccgaggcggccgacatgnnnnnnnnttttttttttttttttttttttttttttttvn

SEQ ID NO: 26 - DNA, 33 bp, synthetic construct
  misc_feature 1..33: note = Synthetic: S-TSO
  modified_base 1: mod_base = OTHER; note = 5'biotin modification, 5Bios
  variation 20..27: note = n is a, c, g, or t
  source 1..33: mol_type = other DNA
rgagacagattgcgcaatgnnnnnnnnrgrgrg

SEQ ID NO: 27 - DNA, 19 bp, synthetic construct
  misc_feature 1..19: note = Synthetic: S3-PCR1-forward primer
  source 1..19: mol_type = other DNA
agagacagattgcgcaatg

SEQ ID NO: 28 - DNA, 39 bp, synthetic construct
  misc_feature 1..39: note = Synthetic: S3-PCR2-forward primer
  source 1..39: mol_type = other DNA
actcgcgagggacgtgaagcagagacagattgcgcaatg

SEQ ID NO: 29 - DNA, 33 bp, synthetic construct
  misc_feature 1..33: note = Synthetic: W-TSO
  modified_base 1: mod_base = OTHER; note = 5'biotin modification, 5Bios
  source 1..33: mol_type = other DNA
rttctagaggccgaggcggccgacatgrgrgrg

SEQ ID NO: 30 - DNA, 27 bp, synthetic construct
  misc_feature 1..27: note = Synthetic: W-PCR1-forward primer
  source 1..27: mol_type = other DNA
attctagaggccgaggcggccgacatg

SEQ ID NO: 31 - DNA, 77 bp, synthetic construct
  misc_feature 1..77: note = Synthetic: W-PCR2-forward primer
  variation 21..50: note = n at positions 21 - 24 must be present; and n at positions 25 - 50 can be present or absent
  variation 21..50: note = n, if present, is a, c, g, or t
  variation 25..50: note = position may be absent
  source 1..77: mol_type = other DNA
actcgcgagggacgtgaagcnnnnnnnnnnnnnnnnnnnnnnnnnnnnnnattctagaggccgaggcggccgacatg

SEQ ID NO: 32 - DNA, 24 bp, synthetic construct
  misc_feature 1..24: note = Synthetic: N-TSO
  modified_base 1: mod_base = OTHER; note = 5'biotin modification, 5Bios
  source 1..24: mol_type = other DNA
rctatctagagcggccgcrgrgrg

SEQ ID NO: 33 - DNA, 18 bp, synthetic construct
  misc_feature 1..18: note = Synthetic: N-PCR1-forward primer
  source 1..18: mol_type = other DNA
actatctagagcggccgc

SEQ ID NO: 34 - DNA, 68 bp, synthetic construct
  misc_feature 1..68: note = Synthetic: N-PCR2-forward primer
  variation 21..50: note = n at positions 21 - 24 must be present; and n at positions 25 - 50 can be present or absent
  variation 21..50: note = n, if present, is a, c, g, or t
  variation 25..50: note = position may be absent
  source 1..68: mol_type = other DNA
actcgcgagggacgtgaagcnnnnnnnnnnnnnnnnnnnnnnnnnnnnnnactatctagagcggccgc

SEQ ID NO: 35 - DNA, 39 bp, synthetic construct
  misc_feature 1..39: note = Synthetic: B-TSO
  modified_base 1: mod_base = OTHER; note = 5'biotin modification, 5Bios
  source 1..39: mol_type = other DNA
rcactctttccctacacgacgctcttccgatctrgrgrg

SEQ ID NO: 36 - DNA, 33 bp, synthetic construct
  misc_feature 1..33: note = Synthetic: B-PCR1-forward primer
  source 1..33: mol_type = other DNA
acactctttccctacacgacgctcttccgatct

SEQ ID NO: 37 - DNA, 53 bp, synthetic construct
  misc_feature 1..53: note = Synthetic: B-PCR2-forward primer
  source 1..53: mol_type = other DNA
actcgcgagggacgtgaagcacactctttccctacacgacgctcttccgatct

SEQ ID NO: 38 - DNA, 29 bp, synthetic construct
  misc_feature 1..29: note = Synthetic: K-TSO
  modified_base 1: mod_base = OTHER; note = 5'biotin modification, 5Bios
  source 1..29: mol_type = other DNA
kagaggccgaggcggccgacatgrgrgrg

SEQ ID NO: 39 - DNA, 23 bp, synthetic construct
  misc_feature 1..23: note = Synthetic: K-PCR1-forward primer
  source 1..23: mol_type = other DNA
tagaggccgaggcggccgacatg

SEQ ID NO: 40 - DNA, 43 bp, synthetic construct
  misc_feature 1..43: note = Synthetic: K-PCR2-forward primer
  source 1..43: mol_type = other DNA
actcgcgagggacgtgaagctagaggccgaggcggccgacatg